(19) United States
(12) Patent Application Publication
(10) Pub. No.: US 2011/0165595 A1
(43) Pub. Date: Jul. 7, 2011

(54) APPARATUS AND METHODS FOR PROCESSING A WHOLE BLOOD SAMPLE

(76) Inventors: Brian E. Catanzaro, San Diego, CA (US); Theodore B. Hill, San Diego, CA (US); Maya Kotob-Yahfoufi, San Diego, CA (US); Christopher J. Johnson, San Diego, CA (US); Kent Gandola, San Diego, CA (US)

(21) Appl. No.: 12/835,524

(22) Filed: Jul. 13, 2010

Related U.S. Application Data
(60) Provisional application No. 61/225,182, filed on Jul. 13, 2009.

(51) Int. Cl. G01N 33/567; C12Q 1/02; C12M 1/38

(52) U.S. Cl. 435/7.21; 435/29; 435/286.1

(57) ABSTRACT

The present invention provides a device and method for heating a whole blood sample for use in an assay, comprising irradiating said whole blood sample with monochromatic visible light of between 380 nm and 740 nm to raise the temperature of said whole blood sample to a target temperature between about 35° C. and 40 ° C. An apparatus for heating whole blood samples stored in a cartridge is further described comprising a housing assembly disposed to receive the cartridge one or more visible light optical emitters disposed in the housing apparatus and configured to selectively illuminate a staging area of the cartridge a temperature sensor disposed in the housing apparatus and configured to sense a temperature of the cartridge and a control circuit coupled to the temperature sensor and the array of visible light optical emitters so as to control a heating profile of the whole blood sample.

### RELATED APPLICATION

This application claims the benefit of U.S. Provisional Application No. 61/225,182, filed on Jul. 13, 2009, which is hereby incorporated by reference in its entirety.

### FIELD OF THE INVENTION

This invention relates generally to the field of diagnostic assays and the collection and processing of samples therefore. More specifically but not exclusively, the present invention relates to apparatus and methods for processing a whole blood sample, including receiving the sample and optically heating the sample to a predefined temperature range.

### BACKGROUND

Whole blood assays are an important tool in clinical care. As an example, the evaluation of platelet functionality when performed on whole blood, rather than blood serum (platelet rich plasma) provides certain advantages in terms of convenience, repeatability, and accuracy.

Once withdrawn from the body, whole blood can change temperature rapidly. Given a core body temperature of 37° C. and an ambient laboratory or clinical environment of 18-21° C., a test tube size sample can cool several degrees in a few minutes.

Performing an assay on blood requires that the sample remain at constant temperature for several reasons. Chemicals, constituents and reagents are often temperature-sensitive. The temperature of the reactants can increase or decrease reaction time causing a significant deviation from in-vivo conditions. In some cases, excessively high temperature can denature components to the point where reactants can no longer even participate in the assay reaction of interest. The accuracy of the temperature set point of the assay, the upper and lower bounds on temperature during, before, and after the assay, and the variation in temperature throughout the volume of the reaction chamber may all play important roles in any given assay.

In addition to the chemical and biological constraints of the assay of interest, regulatory bodies have established requirements for the temperature of blood during diagnostic tests. The temperature requirement prescribed by the College of American Pathologists (CAP) is currently 37° C.

The design of point-of-care instruments must not only take into account these temperature requirements, but also the economic and ease of use concerns of the marketplace and user(s). The economics of healthcare drives instrumentation to be low cost. This implies that reaction chambers are rarely cleaned, disinfected, and re-used; they are more often disposed. Requirements for ease-of-use also encourage instrument designs that involve single-use reaction chambers. Disposable reaction chambers or assay devices reduces the cost of the process by eliminating cleaning as well as providing assurance that reactions occur without contamination. The materials currently used in disposable designs often are not efficient for heat transfer.

Various technical and market concerns prohibit designs where the assay device takes several minutes to achieve the required temperature conditions. In many cases, the assay must be performed in minutes to provide results that are useful to healthcare providers.

In summary, the marketplace requires an easy-to-use instrument that must perform an assay in an accurate, isothermal condition. However, the sample that is introduced to the instrument is often much cooler than necessary to perform the assay. Consequently, the sample must be rapidly and accurately heated prior to performing the assay.

Materials of choice for assay devices which contain the sample while the assay is performed are often plastics or silica based glass (e.g., borosilicate, quartz, BK7) materials. These materials have the distinct disadvantage when heating in that their thermal conductivity is low and their diffusivity also is low. The thermal conductivity determines the difference in temperature between the inside and outside wall of the assay device. Low thermal conductivity exhibits a large temperature difference (drop) from the outside of the assay device to the inside of the assay device where the blood sample is stored during the assay. Diffusivity determines the rate at which heat flows through the material and the rate at which it reaches equilibrium. Therefore, low thermal diffusivity materials take a long time to heat. Consequently, common materials for assay devices take a long time to heat and exhibit a large temperature difference (gradient) between the exterior surface and the interior surface where the assay sample is stored.

A convenient method of heating the assay device is by conduction; that is, by placing the assay device in contact with an object at the desired temperature (e.g. a hot plate at 37° C.). However, the materials of construction for the assay device are such that in many situations, this approach results in a very long heating time.

The approach of increasing the temperature of the hot plate above 37° C. to increase the rate of heating is not preferred. It has two drawbacks. First, the control algorithm is difficult to achieve. Heat can get stored in the inside of the assay device that will later transfer to the sample in a complex manner. Furthermore, assays and samples usually have a stringent maximum temperature requirement. Exceeding the maximum temperature invalidates the results of the assay. The approach to increase the temperature of the hot plate above 37° C. incurs the risk that the assay constituents and/or sample may exceed their maximum temperature and begin to denature.

Accordingly, there is a need in the art for alternate approaches to heating a sample to avoid the above problems as well as provide other potential advantages.

### SUMMARY

In one embodiment, the present invention provides a method for heating a whole blood sample for use in an assay, comprising irradiating said whole blood sample with narrowband visible light of between 380 nm and 740 nm until the temperature of said whole blood sample is blood sample between about 35° C. and 40° C.

In another aspect, the present invention provides a method for determining platelet binding function in a whole blood sample. The method comprises irradiating the whole blood sample with narrowband or monochromatic visible light of between 380 nm and 740 nm until the temperature of said whole blood sample is between about 35° C. and 40° C.; combining the whole blood sample with an agglutinating system to form an assay mixture; irradiating the assay mixture with infrared light, determining the optical transmittance of the assay mixture to infrared light; wherein the optical transmittance or a change in the optical transmittance over time is an indication of binding function of the platelets in the whole blood sample.

In one aspect, the present invention is directed to an apparatus for heating a whole blood sample in a cartridge, comprising a housing assembly disposed to receive the cartridge, an array of optical emitters disposed in the housing apparatus and configured to selectively illuminate a staging area of the cartridge with narrowband or monochromatic visible light, a temperature sensor disposed in the housing apparatus and configured to sense a temperature of the staging area of the cartridge and a control circuit coupled to the temperature sensor and the array of optical emitters so as to control a heating profile of the whole blood sample.

In another aspect, the present invention is directed to a system for testing whole blood comprising a cartridge disposed to store the whole blood sample in a staging area and a testing apparatus including a housing assembly disposed to receive the cartridge, an array of optical emitters disposed in the housing apparatus and configured to selectively illuminate the staging area with narrowband or monochromatic visible light so as to heat the whole blood sample, a temperature sensor disposed in the housing apparatus and configured to sense a temperature of the cartridge and a control circuit coupled to the temperature sensor and the array of optical emitters so as to control said heating of the whole blood sample in order to attain a target temperature.

In another aspect, the present invention is directed to a processor readable medium comprising instructions for execution on a processor to determine an initial temperature of a whole blood sample, optically heat, responsive to said determining, the whole blood sample, determine an additional temperature of the whole blood sample, compare said additional temperature to a target temperature and repeat said heating, determining and comparing until said additional temperature reaches the target temperature.

Additional aspects of the present invention are further described herein in conjunction with the appended drawings.

### BRIEF DESCRIPTION OF THE DRAWINGS

FIG. 1 is a diagrammatic plan view of a device according to the subject invention.

FIG. 2 is a plan view of an assembled device of FIG. 1.

FIG. 3 is a diagrammatic plan view of the device of FIG. 2 without a cover plate.

FIG. 4 is an exploded view of the device of FIG. 1.

FIG. 5 is diagrammatic plan view of an alternative embodiment of a device in accordance with the present invention.

FIG. 6 is a schematic diagram of one embodiment of an instrument that may be employed in conjunction with a device in accordance with the present invention.

FIG. 7 is a diagrammatic plan view of an alternate embodiment of a device according to the subject invention.

FIG. 8a illustrates an embodiment of a whole blood testing system in accordance with aspects of the present invention.

FIG. 8b illustrates an embodiment of a heating sub-assembly for the whole blood testing system illustrated in FIG. 8a.

FIGS. 9a and 9b illustrates the absorption characteristics for different visible light wavelengths in whole blood.

FIG. 9c illustrates absorption of light by whole blood over a range of visible light wavelengths.

FIG. 9d illustrates the optical transmittance characteristics of various materials at Mid-Wavelength Infra-Red (MWIR) wavelengths.

FIG. 9e illustrates additional details of FIG. 9d.

FIGS. 10a and 10b illustrate an embodiment of an optimized LED emitter array layout based on a predefined staging area configuration.

FIG. 11 illustrates an embodiment of process workflow for heating whole blood in an assay system.

FIG. 12 illustrates an embodiment of a process workflow for selecting optical components and determining an optimized layout so as to provide uniform heating to a sample.

### DETAILED DESCRIPTION OF EMBODIMENTS

As note previously, there are a variety of problems associated with heating biological fluid samples such as whole blood using a heat plate or immersion in an assay system. In contrast to conductive heating approaches, the approach of radiation as a mode of heat transfer is appealing because the energy from the radiation source is directly deposited in the sample of interest. Assuming that the assay device is transparent to the radiation and the sample absorbs the radiation, the sample is heated directly with no energy or time wasted heating the assay device. The requirements are that the assay device and light source must be chosen such that the assay device transmits radiation that will be absorbed by the sample.

When considering radiation as a method of heat transfer, one additional parameter should be considered: the thermal conductivity and diffusivity of the sample itself. If the absorption coefficient of the blood sample is high, then all of the optical radiation is absorbed at the surface of the sample (e.g. within a few microns or millimeters). This may often result in the exterior surface of the sample achieving the required temperature while the interior remains colder than desired. An ideal situation would be a moderate absorption coefficient, distributing the energy throughout the volume of the sample.

Whole blood has many different absorption bands. The ultraviolet and shorter spectrum can damage proteins, whole blood constituents (e.g. red blood cells, white blood cells, platelets), and assay reagents. The infrared spectrum can be limited in its practicality by the transmittance spectrum of assay devices (e.g. glass and plastic) as well as the availability of low cost sources. Visible sources would first appear to be a poor choice, given the relatively high transmittance of whole blood and plasma to visible light. However, at suitable sample thickness, hemoglobin exhibits sufficient absorbance to convert virtually all of the incident visible light into thermal energy.

An additional consideration in the interaction of whole blood samples and light is the degree to which visible light is scattered. The microstructure of the constituents in whole blood results in a relatively short mean transport length of a visible photon. Unlike homogenous fluids, whole blood is more similar to a suspension. Particles (corpuscles) suspended in a fluid have a tendency to scatter light. Therefore, incident light (photons) through macroscopic thickness of whole blood do not take a straight path. Photons are scattered by the particles (and their structure) suspended in the fluid. This increases the actual path length of a photon traveling through the fluid. A photon traveling straight through a homogenous fluid will have a path length equal to the thickness of the fluid. Therefore, the absorbance is related to the absorption coefficient and the actual thickness of the fluid. A photon incident on a scattering medium (e.g. whole blood) takes a much more convoluted path as it is redirected by scattering off of various objects suspended in the fluid. This process increases the interaction length in the sample and makes the effective absorption of light even for small absorption coefficients higher.

It is useful to incorporate a feedback mechanism to control the illumination of the sample. When placing a sample in contact with a hot plate, the sample cannot exceed the temperature of the hot plate. However, when using radiation heat transfer, the sample can be heated to temperatures much hotter than the surrounding (transparent) medium. Consequently, some means must be used to control the amount of radiation. At a minimum a timer must be used. More effective is the use of a sensor and control mechanism to limit the rate of increase of temperature of the sample and the absolute temperature of the sample.

The present invention provides a method for heating a whole blood sample for use in an assay, comprising irradiating said whole blood sample with narrowband or monochromatic visible light of between 380 nm and 740 nm until the temperature of said whole blood sample is between about 35° C. and 40° C. The present invention also provides apparatus for controlled heating of a blood sample within an assay system or other system requiring similar heating requirements. In general, heating of whole blood (or other blood or biological fluids) in assay applications such as those described herein can be characterized by a heating profile. In such a heating profile, the blood is received from a sample of blood taken from a patient or from a stored blood sample collected previously from the patient, and then introduced to the assay system for analysis. The analysis typically must be performed at a predefined target temperature within a predefined time.

Before proceeding further with a detailed description of the present invention, a number of terms as used herein are defined.

As used herein the term “initial temperature” refers to the temperature of the whole blood sample prior to being heated by irradiation with visible light. Typically, the whole blood sample will be heated from an initial temperature to a target temperature by continuous irradiation with visible light. When the whole blood sample reaches a target temperature, the visible light irradiation is permanently discontinued. Alternatively, the whole blood sample may be heated to a desired target temperature with intermittent irradiation with visible light.

As used herein the term “monochromatic” refers to light of a wavelength spectrum of less than or equal to 100 nm. For example, visible light having a spectrum from 600 nm to 700 nm is monochromatic because it includes only wavelengths within a 100 nm range. Conversely, visible light having a spectrum from 550 nm to 700 nm is not monochromatic because its wavelength spectrum is 150 nm, and is therefore greater than 100 nm.

As used herein the term “colored visible light” refers to visible light of less than the entire visible light spectrum. Thus, colored visible light irradiation includes irradiation of a single color of the visible light spectrum as well as irradiation that includes more than one color of the visible light spectrum. For example, irradiation with visible light in both the blue and red regions is irradiation with colored visible light.

As used herein the visible light spectrum is defined as follows:

Visible Light Spectrum

| Color | Wavelength (nm) |
|---|---|
| Red | 625-740 |
| Orange | 590-625 |
| Yellow | 565-590 |
| Green | 520-565 |
| Cyan | 500-520 |
| Blue | 435-500 |
| Violet | 380-435 |

As used herein the term “high output LED” refers to an LED device that operates at currents substantially higher than standard LED devices and provides correspondingly higher optical output power above that of standard LED products. For example, currently available standard output LEDs operate at approximately 30 mA and will fail at approximately 50 mA. High output LEDs nominally operate at approximately 700 mA and will fail at approximately 1000 mA, or about an order of magnitude higher than standard LED devices.

Sample—any solution, synthetic or natural, containing an analyte, including body fluids such as, for example, whole blood, blood fractions such as serum and plasma, synovial fluid, cerebro-spinal fluid, amniotic fluid, semen, cervical mucus, sputum, saliva, gingival fluid, urine, and the like, and aqueous or water soluble solutions of natural or synthetic compounds, particularly, compounds that are potential therapeutic drugs, and it is desired to determine if the compound binds to a specific receptor. The amount of the sample depends on the nature of the sample and the analyte contained therein. For fluid samples such as whole blood, saliva, urine and the like the amount of the sample is usually about 0.1 to 10 ml, more usually, about 1.8 to 4.5 ml. The term “sample” includes unprocessed samples directly from a patient or samples that have been pretreated and prepared in any convenient medium although an aqueous medium is preferred.

As mentioned above, the sample may be preprocessed prior to placing the sample in a sample container or cartridge. Such preprocessing may include lysing of cells in the sample, releasing an analyte from binding materials in a sample, absorbing unwanted materials by affinity matrices, and so forth. Reagents for lysing cells in the sample include, for example, ammonium chloride, sodium chloride, detergents such as Triton X-100, Zwittergen and the like. The amount of the lysing reagent applied to the sample is generally sufficient to bring about the level of lysing desired and is usually about 0.01 to 10% by weight.

Other preprocessing reagents include precipitation reagents, affinity matrices with antibodies or antigens or lectins and so forth. The amount of such a reagent employed is generally sufficient to achieve the desired result and the reagent is applied in a manner similar to that described above for the stabilization reagent.

In one embodiment, the present invention provides a method for heating a whole blood sample for use in an assay, comprising irradiating the whole blood sample with monochromatic visible light of between 380 nm and 740 nm until the temperature of the whole blood sample is between about 35° C. and 40° C.

In one embodiment, the present invention provides a method for heating a whole blood sample for use in an assay, comprising irradiating the whole blood sample with colored visible light of between 380 nm and 740 nm until the temperature of the whole blood sample is between about 35° C. and 40° C.

In one embodiment of the present invention the whole blood sample is irradiated with visible light of between 600 nm and 680 nm. In another such embodiment, the whole blood sample is irradiated with visible light of between 610 nm and 660 nm. In still another such embodiment, the whole blood sample is irradiated with visible light of between 615 nm and 650 nm. In yet another such embodiment, the whole blood sample is irradiated with visible light of between 620 nm and 645 nm.

In one embodiment of the present invention the irradiation from the monochromatic visible light raises the temperature of the whole blood sample to a target temperature between about 36° C. and 38° C. In one such embodiment, the irradiation from the monochromatic visible light raises the temperature of the whole blood sample to a target temperature between 36.5° C. and 37.5° C. In another such embodiment, the irradiation from the monochromatic visible light raises the temperature of the whole blood sample to a target temperature of about 37° C.

In one embodiment of the present invention the irradiation is emitted by one or more light emitting diodes (LEDs), however, other light sources capable of efficiently providing relatively narrowband light output at visible wavelengths may alternately be used. For example, other sources may include incandescent filament light bulbs with filters, gas discharge lamps (e.g., arc lamps), fluorescent sources with appropriate filters or phosphors, plasma emitters, lasers, or other devices capable of emitting energy at optical wavelengths. In one such embodiment, the irradiation is emitted by one or more light emitting diodes. In one such embodiment, the one or more light emitting diodes are high brightness LEDs. In one such embodiment, the irradiation is emitted by an array of light emitting diodes. In addition, the array may be further divided into two or more sub-array such that each sub-array illuminates a portion of the container or cartridge used to store the sample. This may be advantageous in embodiments where the container or cartridge can be illuminated from multiple surface simultaneously so as to provide more uniform heating to the sample. In some embodiments, multiple LEDs may be used, where individual LEDs are controlled separately so as to modify the uniformity of the illumination. In addition, in some embodiments using multiple LEDs, the individual LEDs may be configured to illuminate at different wavelengths or ranges of wavelengths.

In one embodiment of the present invention the initial temperature of the whole blood sample is between about 18° C. and 34° C. In one such embodiment, the initial temperature of the whole blood sample is between about 18° C. and 25° C. In another such embodiment, the initial temperature of the whole blood sample is between about 21° C. and 25° C.

In one embodiment of the present invention the irradiation from the monochromatic visible light raises the temperature of the whole blood sample to a target temperature of about 37° C. within 180 seconds. In one such embodiment, the irradiation from the monochromatic visible light raises the temperature of the whole blood sample to a target temperature of about 37° C. within 120 seconds. In another such embodiment, the irradiation from the monochromatic visible light raises the temperature of the whole blood sample to a target temperature of about 37° C. within 90 seconds. In still another such embodiment, the irradiation from the monochromatic visible light raises the temperature of the whole blood sample to a target temperature of about 37° C. within 60 seconds. In yet another such embodiment, the irradiation from the monochromatic visible light raises the temperature of the whole blood sample to a target temperature of about 37° C. within 30 seconds.

In one embodiment of the present invention the method for heating a whole blood sample for use in an assay comprises determining the initial temperature of the whole blood sample.

In one embodiment of the present invention the method for heating a whole blood sample for use in an assay comprises determining the target temperature of the whole blood sample. In one such embodiment, the target temperature of the whole blood sample is determined using an optical sensor.

In one embodiment of the present the thickness of the whole blood sample is between about 1 mm and 5 mm. In one such embodiment, the thickness of the whole blood sample is between about 1 mm and 2 mm. In another such embodiment, the thickness of the whole blood sample is between about 2 mm and 3 mm. In another such embodiment, the thickness of the whole blood sample is between about 3 mm and 4 mm. In yet another such embodiment, the thickness of the whole blood sample is between about 4 mm and 5 mm. In one particular embodiment, the thickness of the whole blood sample is about 3.6 mm.

In another aspect, the present invention provides a method for determining platelet binding function in a whole blood sample. The method comprises irradiating the whole blood sample with monochromatic visible light of between 380 nm and 740 nm to raise the temperature of the whole blood sample to a target temperature between about 35° C. and 40° C.; combining the whole blood sample with an agglutinating system to form an assay mixture; irradiating the assay mixture with infrared light, determining the optical transmittance of the infrared light through the assay mixture; wherein the optical transmittance or a change in the optical transmittance over time is an indication of binding function of the platelets in the whole blood sample. In one embodiment, the steps following irradiation of the whole blood sample with monochromatic visible light may be performed in accordance with the procedures described in U.S. Pat. No. 5,922,551, the entire contents of which are expressly incorporated by reference herein.

In another aspect, the present invention provides an apparatus for heating a whole blood sample in a cartridge, comprising a housing assembly disposed to receive the cartridge, an array of optical emitters disposed in the housing apparatus and configured to selectively illuminate a staging area of the cartridge, a temperature sensor disposed in the housing apparatus and configured to sense a temperature of the cartridge and a control circuit coupled to the temperature sensor and the array of optical emitters so as to control a heating profile of the whole blood sample.

In another aspect, the present invention provides a system for testing whole blood comprising a cartridge disposed to store the whole blood sample in a staging area and a testing apparatus including a housing assembly disposed to receive the cartridge, an array of optical emitters disposed in the housing apparatus and configured to selectively illuminate the staging area so as to heat the whole blood sample, a temperature sensor disposed in the housing apparatus and configured to sense a temperature of the cartridge and a control circuit coupled to the temperature sensor and the array of optical emitters so as to control said heating of the whole blood sample in order to attain a target temperature.

In another aspect, the present invention comprises a processor readable medium comprising instructions for execution on a processor to determine an initial temperature of a whole blood sample, optically heat, responsive to said determining, the whole blood sample, determine an additional temperature of the whole blood sample, compare said additional temperature to a target temperature and repeat said heating, determining and comparing until said additional temperature reaches the target temperature.

One aspect of the invention concerns a device for receiving and processing a sample. The device comprises a sample receiving element adapted to establish fluid communication with and receive a sample directly from a sample container. The sample container is usually a container in which the sample to be processed is collected. The sample container may be in any form such as a syringe, test tube, cuvette, vial, cartridge and the like. For blood samples the sample container may conveniently be a Vacutainer® container, a syringe and so forth. Suitable materials for fabrication of the sample container are glass, plastic and the like. In general, any material may be used that does not react with, or otherwise cause detrimental effects on, the sample or any solvents in which the sample is dissolved or suspended. The sample container may not necessarily be a container in which sample is collected. For example, the sample container may be a container in which a sample is placed after collection and pre-processing such as to remove debris, filter cells, add diluents and so forth.

An appropriate element is included as part of the sample container for attachment to the sample receiving element, such as a container or cartridge, of a device in accordance with the present invention. For instance, if the sample receiving element of the present device includes a needle or other piercing element, the sample container comprises a corresponding element capable of being pierced such as a septum, membrane, and the like. Alternatively, the sample receiving element and the sample container can have other mating elements that provide for sealed fluid communication between the instant device and the sample container. For example, the sample container may include a piercing element and the sample receiving element may comprise a corresponding septum. The primary principle involved is that sample can be transferred from the sample container to the present device without opening the sample container. Other suitable mating elements include Luer fittings and other mechanical sealing connections.

The sample container may include one or more other features depending on the nature of the sample and its processing. For example, separation elements such as filters, membranes and the like may be included. As mentioned above, in addition to establishing fluid communication between the sample container and the present device, the sample receiving element also allows for introduction of a sample into the device. A filter element may be employed for removing particles and other debris from the sample. In one embodiment, where it desired to analyze serum or plasma, the filter element can provide for the efficient removal of red blood cells from a whole blood sample so as to provide a serum or plasma sample substantially free of interfering red blood cells or hemoglobin or metabolic or degradation products thereof. The filter element can also be used to remove particles and other unwanted materials from other types of samples, such as urine and the like.

The sample receiving element may be of any suitable design, preferably a design that provides for holding the sample container when the latter is secured to the instant device. Conveniently, the sample receiving element may be a recess, such as a well or the like, in a housing. In such a configuration part or all of the sample container can be secured in the well. The recess may include friction elements for securing the sample container in the well. The friction elements may take the form of circumferential ribs, longitudinal ribs, spring fingers and so forth. As described above, the sample receiving element also comprises a component for establishing sealed fluid communication with the sample container.

One convenient design for the sample receiving element is a needle assembly comprising a needle and a needle holding means that is attached to the base of the bottom inside wall of a well. The needle holding means generally comprises a cylindrical passageway in a housing in which the needle can be mounted. The device may be manufactured with the needle secured in the housing. On the other hand, the needle can be secured in the housing prior to use. The needle is usually constructed from metal tubing and is usually about 26 to 16 gage. The dimensions of the needle are about 10 to 15 mm, preferably, about 13 mm in length and about 1 to 1.5 mm, preferably, about 1.3 mm, in outside diameter, and about 0.5 to 1 mm, preferably, about 0.75 mm, in inside diameter. The needle holding means can be of any convenient size and shape as long as it holds the needle to permit ready piercing of the sample container. The needle holding means generally has a bore therethrough to provide access of the sample to the device. The needle assembly may include a cover for the needle portion to protect both the needle and the user.

At least one first chamber is in fluid communication with the sample receiving element. Fluid communication may occur through a channel or capillary between the sample receiving element and the first chamber. Generally, the size of the channel or capillary is about 0.1 mm to about 3 mm, more usually, 0.8 to 1.3 mm, in diameter. The size of the first chamber is dependent on the nature of the sample, the suspected concentration of any component to be determined, sample heating time and so forth. Generally, the first chamber is about 0.1 to 5 ml, usually about 0.6 to about 2 ml.

The first chamber serves as a staging area for the sample to be processed. In the first chamber the sample may be processed such as by incubation at a particular temperature or temperatures, exposure to certain processing agents such as, e.g., enzymes, reagents, activators, inhibitors, lysing agents contained in the first chamber, and so forth. It is desirable that the communication between the sample receiving element and the first chamber occur at a point, the fill point, in the first chamber that provides maximum separation between the sample input point into the first chamber and the point at which pressure is adjusted in the first chamber. Furthermore, preferably, the fill point is also remote from the point at which fluid exits the first chamber (exit point). In a preferred embodiment the fill point is at or near the top portion of the first chamber and the exit point is at or near the bottom of the first chamber. Such a configuration maximizes the avoidance of premature filling of the first chamber. Also included is means for preventing premature movement of fluid out of the first chamber. Exemplary of such means are valves either passive or active by external means, resistive elements, capillary stop junctions, and the like.

The device also includes one or more second chambers that are in fluid communication with the first chamber. Fluid communication may occur through a channel or capillary between each of the second chambers and the first chamber. Generally, the size of the channel or capillary is about 0.1 mm to about 3 mm, more usually, 0.8 to 1.3 mm, in diameter. Generally, the first chamber is about 0.1 to 5 ml, usually about 0.6 to about 2 ml.

A detector can be included between the first chamber and the second chambers to monitor movement of the sample into the second chambers prior to the desired time. Premature filling of the second chambers can be detected in this fashion. The detector may take the form of a transmissive or reflective optical sensor, ultrasonic detector and the like. This may also be used as means for accurate measurement of the start time to fill.

The second chambers are used for conducting further processing of the sample. For example, the second chambers can contain various reagents for conducting an assay. A mixing means may be included in the second chambers for mixing the reagents with the sample introduced into the second chambers. A suitable mixing means is a mixing ball or the like. The mixing ball may be made from material susceptible to magnetic influence, such as ferrous material and the like, and caused to move at an appropriate time by application of a magnetic field. In a preferred embodiment the second chambers are constructed so that the results of an assay may be read either optically or mechanically. Accordingly, the second chambers are usually optically transmissive so that signals generated in an assay may be read, for example, when the present device is inserted into an appropriate instrument.

The device also comprises first and second ports. The first port provides for venting the device. The port can be adapted so that it readily connects to a valve for controlling the outlet of air or other gas from the device. In general, the valve permits flow only in one direction. Suitable valves include check valves, solenoid valves, shuttle valves and so forth. Usually, the first port is adapted for ready connection to a valve by mating means such as a compliant fitting, Luer style fitting and the like. The corresponding mating means from the venting valve is generally found at the end of a channel, capillary, or other tubing. The venting valve and its capillary may be part of an apparatus or instrument in which the present device is inserted.

The second port provides for establishing communication between the device and means for moving the sample from the sample receiving element to the first chamber and for moving the sample from the first chamber to the one or more second chambers. One such means is alternately increasing and decreasing pressure in the device. In one embodiment such means comprises a capillary or channel that is branched and forms a loop at one end, thus creating a pneumatic circuit. The second port is at the end of a channel or capillary leading from the first chamber, preferably, from a point adjacent the top of the first chamber and opposite the fill point to provide optimum filling of the first chamber and transfer to the second chambers at an appropriate point in time. Included within the loop are two-three way valves separated by an intervening air pump, which may be, for example, a diaphragm pump, piston, rotary vane pump and the like. If a reversible pump is used, only one two-way valve is required. The valves are connected to the air pump and to the capillary and are configured such that in one position air may be pumped into the device to create pressure and in another position air may be pumped out of the device to create a vacuum. The pneumatic circuit conveniently may be part of an apparatus in which the present device is inserted. Usually, the second port is adapted for ready connection to the capillary from the pneumatic circuit by mating means such as compliant coupling, Luer style fitting and so forth. The corresponding mating means from the pneumatic circuit is generally found at the end of a channel, capillary, or other tubing.

When the present device comprises more than one second chamber, the channel leading from the first chamber to the second chambers is interrupted by a first manifold. The position of the manifold is generally after the detector mentioned above, if such is included in the device. The size of the manifold is dependent upon the volume of the sample to be moved to the second chambers. Usually, the first manifold is about 0.1 to about 3 mm, in diameter, more usually, 0.8 to 1.3 mm. The cross-sectional area of the first manifold may be varied to maintain proper flow characteristics.

Each of the second chambers is connected to the first manifold by a channel or capillary, which connects to the second chamber, preferably, at a point adjacent the bottom of the second chamber. This channel or capillary is configured to prevent any reagents or other materials in the second chambers from contaminating the common areas of the device, particularly, the first manifold mentioned above. To this end, the channels or capillaries may have an S- shape so as to form a J-trap to prevent migration of reagent. Generally, the size of the channels or capillaries is about 0.1 to about 3 mm, more usually, 0.5 to 1 mm, in diameter.

Each of the second chambers is in fluid communication with an exit port that is part of a second manifold. Usually, each second chamber is connected to its respective exit port by means of a channel or capillary. Generally, the size of the channel or capillary is about 0.1 to about 2 mm, more usually, 0.3 to 0.7 mm, in diameter. One end of the channel or capillary is usually connected adjacent to the top of the second chamber.

Also included as part of the device is means for controlling the precise amount of the sample introduced into each of the second chambers. In one embodiment this is achieved by having the point of connection for the channel or capillary connected to the exit port configured with respect to the fill point so that all air within the second chamber is forced out through the exit port. Optimally, such a configuration is achieved by positioning the point of connection diagonally opposed to the fill point. Furthermore, the shape of the second chamber may be chosen to optimize removal of air during filling and controlling the precise amount of sample introduced into the second chambers. To this end, the shape of the second chamber can be selected so that the top of the second chamber slopes upwardly to the point of connection of the channel or capillary leading to the second manifold. The shape of the second chambers, therefore, may be rhomboidal, triangular and the like. In this way precise filling of the second chambers can be realized, which is important for obtaining accurate, reproducible quantitative results in assays.

Each exit port allows air to escape from the second chamber as the chamber fills with sample. The exit port is designed to permit air, but not liquid, to escape from the second chamber. This effect may be achieved in a number of ways. For example, the exit port may be fitted with a material that permits air to pass through but, when liquid contacts the material, a seal is formed. Such materials include, by way of illustration and not limitation, porous polymer, e.g., Porex XM-1374® (from Porex Technologies, Inc., hydrophobic membranes such as, e.g., Gore-Tex® (from W.L. Gore & Associates, Inc.), and the like. Typically, the size of the exit ports is about 0.1 to 0.2 mm, usually about 0.3 to about 0.7 mm. Other means for achieving the above effect include solenoid valves and optical sensors to close off ports. The exit port may also include a detector for detecting when the second chambers are filled. Such detectors include, for example, reflective and transmissive optical detectors, ultrasonic detectors and the like.

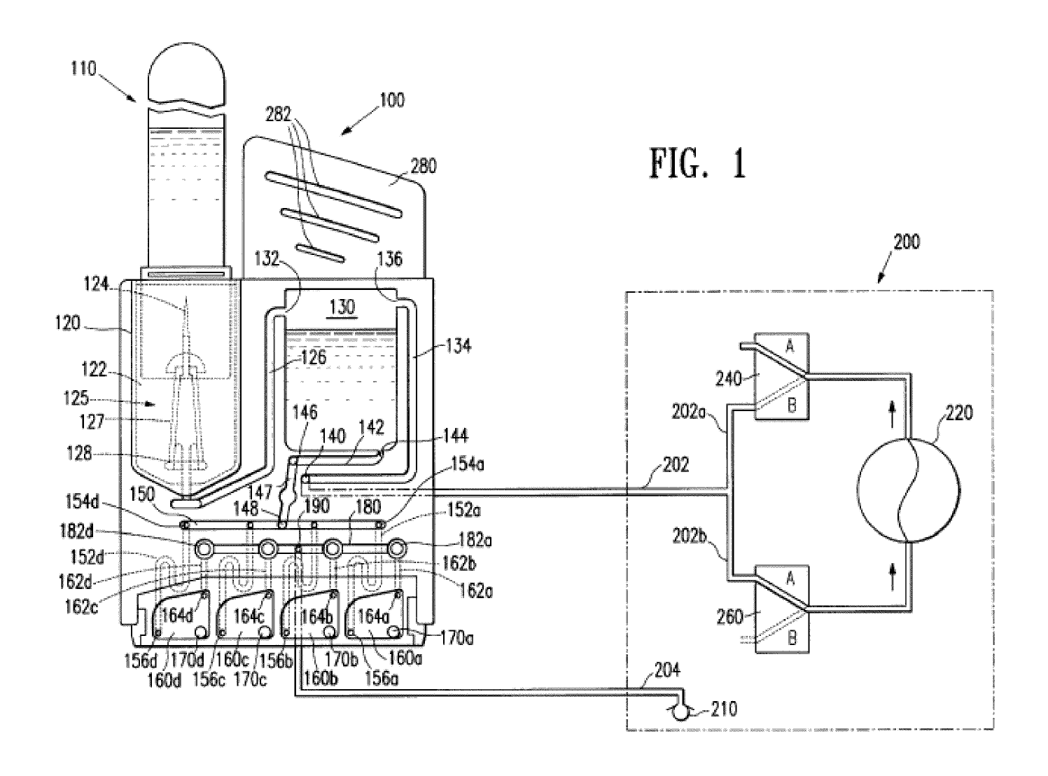

An embodiment of a device in accordance with the present invention is depicted in FIG. 1 by way of illustration and not limitation. Device 100 is shown with a sample container 110 mated with a sample receiving element 120, which comprises well 122. An input needle 124 is part of a needle assembly 125, which comprises needle 124, needle holder 127 and base 128 affixed to the bottom inside wall of well 122. Both needle holder 127 and base 128 comprise a longitudinal bore to provide for fluid to enter device 100. Neelde 124 is in fluid communication with a first chamber 130 by means of a first channel 126, which at one end is connected to base 128 of needle assembly 125 and at the other end to an upper part 132 of first chamber 130. A dual cannula may also be used.

A second channel 134 is connected at one end 136 to first chamber 130 and terminates at the other end at port 140. A third channel 142 provides fluid communication between first chamber 130 and first manifold 150. Accordingly, 142 is connected at its one end 144 to the base of 130 and at its other end 146 to indicator 147. The purpose of indicator 147 is to monitor flow out of first chamber 130 so that premature leakage of fluid from first chamber 130 may be detected. Indicator 147 is connected at point 148 to first manifold 150, thus lying between first manifold 150 and third channel 142 and forming part of the fluid communication between first chamber 130 and first manifold 150.

Fourth channels 152 (152a, 152b, 152c and 152d) provide fluid communication between first manifold 150 and second chambers 160. The device depicted has second chambers 160a, 160b, 160c and 160d. Lying within second chambers 160 are mixing balls 170 (170a, 170b, 170c and 170d, respectively). Fourth channels 152 connect at one end 154 (154a, 154b, 154c and 154d, respectively) to first manifold 150 and at the other end 156 (156a, 156b, 156c and 156d, respectively) to the bottom left of second chambers 160. Diagonally across from 156 are fifth channels 162 connected at one end 164 to the top right corner of 160 and at the other end to second manifold 180 at vent plugs 182 (182a, 182b, 182c and 182d, respectively). Second manifold 180 contains vent port 190.

Device 100 depicted in FIG. 1 is shown in conjunction with pneumatic circuit 200 wherein communication is established between device 100 and pneumatic circuit 200. To achieve such communication, port 240 is connected to sixth channel 202 and vent port 290 is connected to seventh channel 204, which terminates at check valve 210. Sixth channel 202 provides for fluid communication between port 240 and pump 220. Sixth channel 202 branches to give 202a and 202b. Three way valve 240 lies between 202 and pump 220 along 202a and three way valve 260 lies between 202 and pump 220 along 202b. The three-way valves 240 and 260 each have positions A and B. When valves 240 and 260 are in position A, valve 240 is open to the atmosphere and valve 260 is on line with channel 202. Conversely, when valves 240 and 260 are in position B, valve 260 is open to the atmosphere and valve 240 is on line with channel 202.

The device depicted in FIG. 1 also includes holder panel 280 for gripping the device. Holder panel 280 has slots 282, which provide a firmer gripping means as the device is manipulated to secure sample container 110 and to place device 100 in a suitable instrument for connection to a pressure varying apparatus and/or to read the results of an assay.

As mentioned above, the device of this invention is generally useful for the analysis of fluid samples, particularly of physiological fluid samples.

In the embodiment of FIG. 1, a sample in removable sample container 110 (not a part of the invention) is inverted and mounted in well 122 of sample receiving element 120. The top of the sample container has a septum, which is pierced by needle assembly 124 so that fluid may flow into device 100. To induce flow of sample into the device, negative pressure is applied to first chamber 130 via port 140, which is shown attached to exemplary pneumatic circuit 200. As mentioned above, pneumatic circuit 200 comprises an air pump 220 and two three way valves 240 and 260. In use, the valves 240 and 260 are set at positions A to remove air from the first chamber 130 and draw sample into first chamber 130. Sample is then transferred to second chambers 160 by applying positive pressure to first chamber 130 through port 140. This is accomplished by switching the three way valves 240 and 260 to position B. Fluid enters second chambers 160 via first manifold 150. Second chambers 160 optionally contain reagents and mixing balls 170. Fluid fills each of second chambers 160 up to vent plugs 80. Vent plugs 180 permit passage of air but not of liquid. Air passes through vent port 190 and out check valve 210.

The device may be fabricated from individual injection molded parts or by any other convenient process. The device may be fabricated from a material that is not reactive with the sample to be analyzed or the processing reagents employed. Furthermore, the material must be able to withstand the temperatures employed in a processing of the sample. In general, any material may be used that does not react with, or otherwise cause detrimental effects on, the sample or any solvents in which the sample is dissolved or suspended. Suitable materials for the manufacture of the present device include, for example, polystyrene, acrylonitrile-butadiene-styrene (ABS), styrene-acrylonitrile (SAN), polyethylene terephthalate (PET), polycarbonate and so forth.

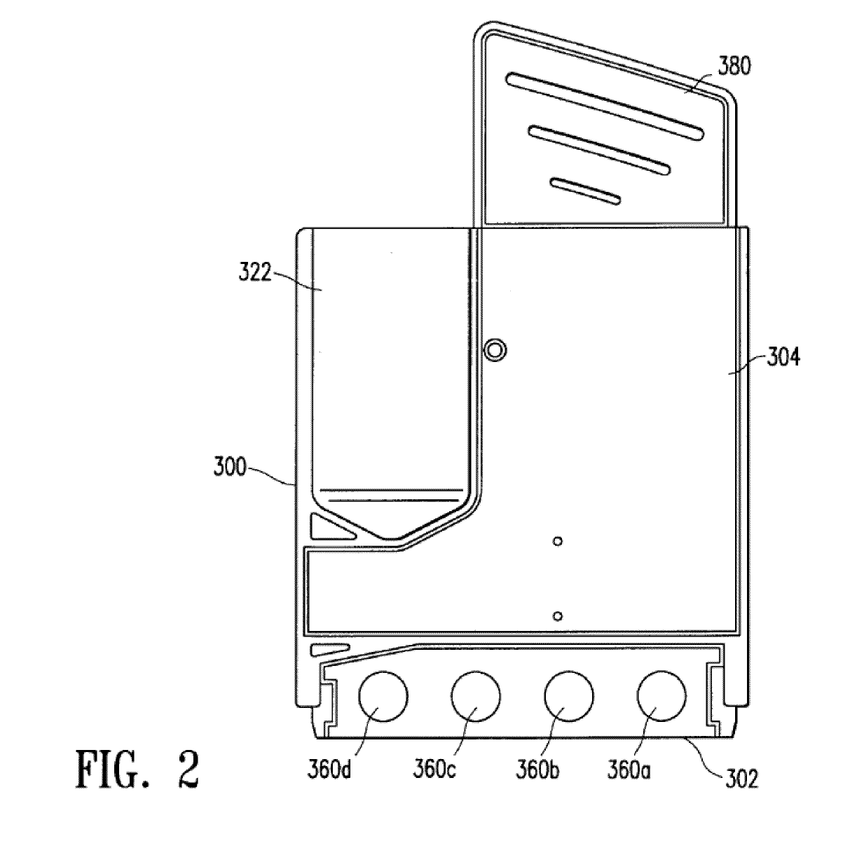

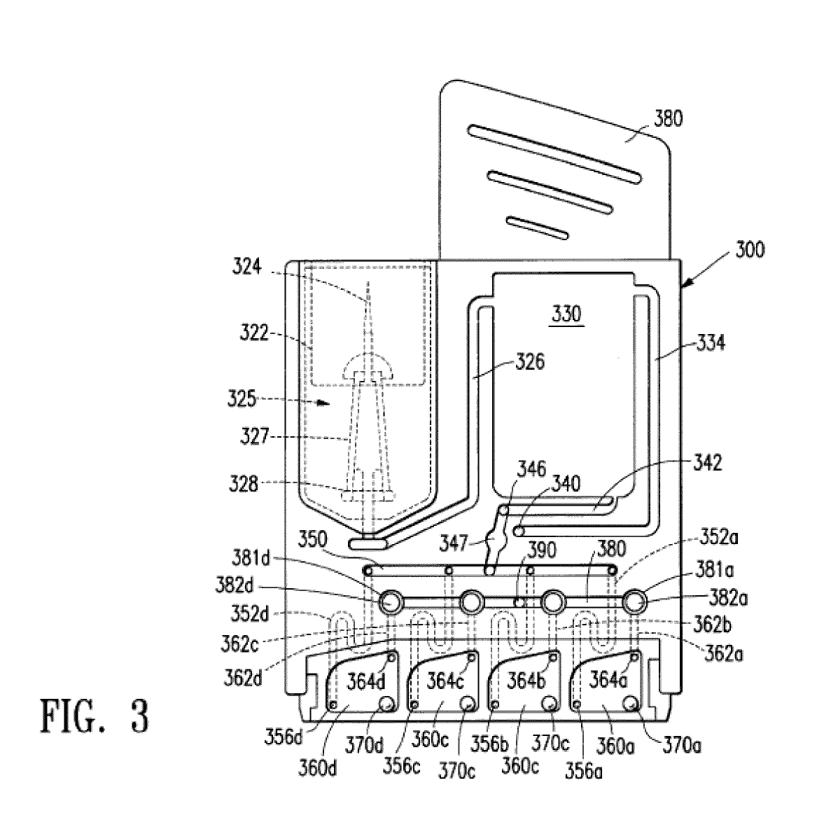

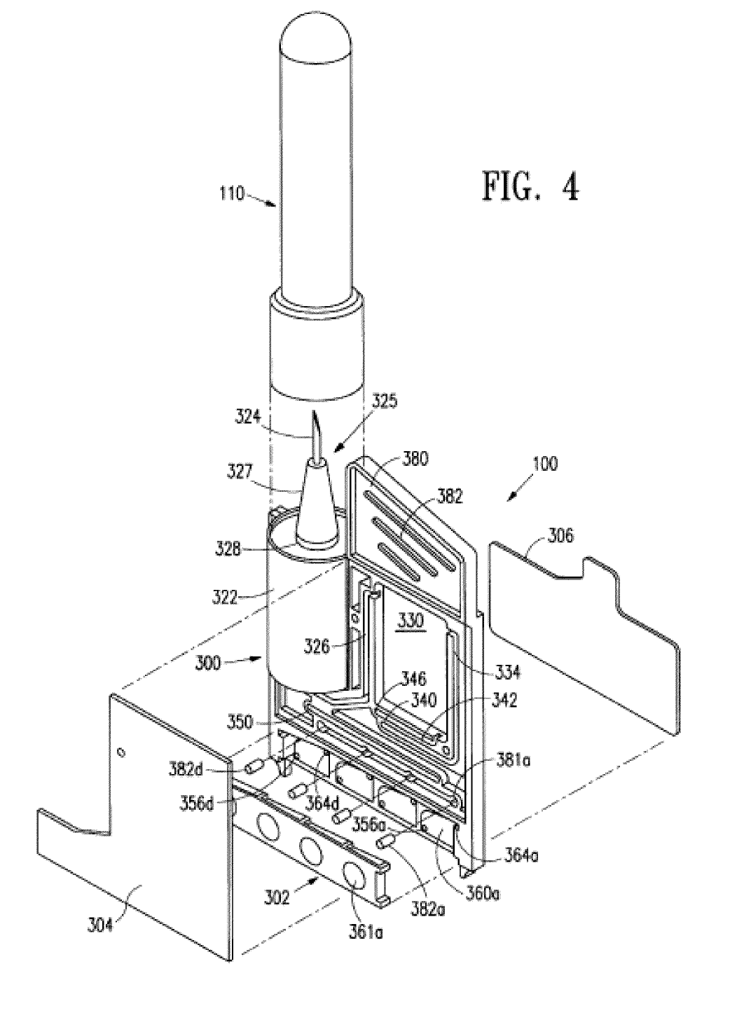

For further understanding of fabrication of a device in accordance with the present invention, by way of example and not limitation, reference is made to FIGS. 2-4. The device depicted is that shown in FIG. 1. There are four individual parts for this embodiment of the present device, namely, housing assembly plate 300, second chamber assembly plate 302, cover plate 304 and rear plate 306. Housing assembly plate 300 includes well 322, which is preformed in housing assembly plate 300. Also preformed in the bottom of well 322 is base 328 and needle holder 327, which are part of needle assembly 325. Needle 324 may be secured in needle holder 327. First channel 326, first chamber 330, second channel 334 including first port 340, third channel 342, indicator 347, first manifold 350, fourth channels 352 (352a, 352b, 352c and 352d), fifth channels 362 (362a, 362b, 362c and 362d), second manifold 380, vent port 390, and vent plug recesses 381 (381a, 381b, 381c and 381d) are all included in housing plate 300. Vent plugs 382 (382a, 382b, 382c and 382d) are placed in vent plug recesses 381. Second chamber plate 302 comprises a cover for second chambers 360 (360a, 360b, 360c and 360d). Second chambers 360 having appropriate openings for aligning with fourth channels 352 at ends 356 (356a, 356b, 356c and 356d). Second chamber plate 302 has appropriate openings for aligning with fifth channels 362 at ends 364 (364a, 364b; 364c and 364d). Housing plate 300 includes a recessed area for inserting second chamber plate 302, which is placed in device 300 so that the openings in the second chambers align with ends 356 and 364. Mixing balls 370 are placed in the second chambers of second chamber plate 302 prior to welding to housing plate 300. Plate 302 is then welded to secure it to housing plate 300. Finally, cover plate 304 and rear plate 306 are welded into position on housing plate 300, thereby completing the manufacture of the device 100.

The primary factor in determining the size of the device is the ease of use of such device. The device should not be so large or so small as to be cumbersome or difficult to use. Furthermore, the size of the device should be such that it is easily manipulated to insert the sample container and to insert the device into an apparatus that has the aforementioned pneumatic circuit as well as a reading means for determining the result of an assay.

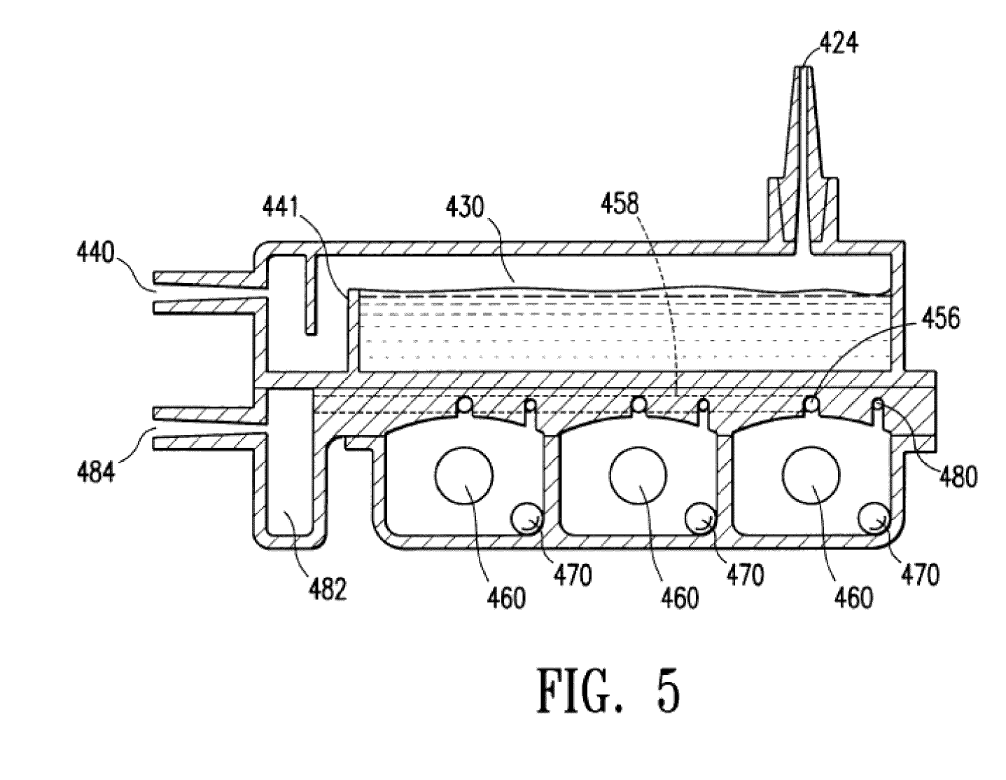

Another embodiment of a device in accordance with the present invention is depicted in FIG. 5. In the device of FIG. 5 fluid is drawn through sample inlet port 424 into first chamber 430 via application of vacuum at port 440. Dam 441 prevents direct flow into the vacuum port 440. The application of positive pressure to port 440 forces fluid into second chambers 460, with mixing balls 470, through fill ports 480, which are in fluid communication with first chamber 430 by means of a manifold (not shown. Vent plugs 456 prevent overflow of fluid while permitting passage of air. Overflow well 482 is connected to check valve connection 484 and to vent plugs 456 by means of manifold 458.

As mentioned above, the device may include one or more reagents for processing the sample. The nature of the reagents for processing the sample will depend on the type of processing to be carried out. Such processing reagents may include reagents for stabilizing and/or preserving the sample or the analyte contained therein and may be included in the first chamber. If the sample is to be subjected to an assay, the nature of the reagents depends on the nature of the assay to be conducted. For example, if the sample is to be analyzed by conducting an immunoassay, the second chamber may include an antibody reagent.

The reagents for processing a sample may include one or more stabilization reagents and/or preservatives for stabilizing and preserving the sample and/or the analyte, applied to the device. Examples of stabilization reagents are chelating compounds such as ethylenediaminetetraacetic acid, water soluble polymers such as polyethylene glycol, polyvinyl pyrrolidine, polyvinyl alcohol, and the like, protease inhibitors such as aprotinin, phenyl methyl sulfonyl fluoride (PMSF), and the like. The amount of stabilization reagent employed is, in general, that which would be effective in bringing about the desired stabilization. The stabilization reagent may be present in an amount of about 0.01 to 2% by weight or more. The stabilization reagent is usually in the form of a buffer containing one or more of the stabilization reagents. Suitable buffers may be any convenient buffer, generally a substantially dilute buffer, which may include phosphate, saline, tris, 3-(N-morpholino)propanesulfonic acid (MOPS), borate, carbonate, or the like. Usually, the buffered solution will be at a pH in the range of about 4 to 9. The buffer concentration is generally from about 10 to 50 mM, preferably, about 15 to 25 mM.

The processing reagents may also include one or more reagents for preserving the sample applied to the device such as to prevent bacterial, fungal and other contamination, e.g., bactericides, antibiotics, fungicides and the like. Such reagents include, for example, sucrose, polyvinyl alcohol, polyvinyl pyrrolidone, dextran, sodium azide, gentamicin, Proclin 300® (Supelco, Bellefonte, Pa.) and so forth. The amount of the preservation reagents employed is about 1 to 20 weight percent, more usually from about 2 to 10 weight percent, and the reagent is applied in a manner similar to that described above for the stabilization reagent.

The processing reagents may include one or more reagents for releasing an analyte from binding proteins and the like that might be present in the sample. Such reagents depend on the nature of the analyte and include, for example, sodium hydroxide, tetrachlorothyronine salicylate, 8-amino-1-naphthalenesulfonic acid, 2-hydroxy-4-methoxybenzophenone-5-sulfonic acid, etc. (see EPA 0 133 464), Nonidet P 40® (NP40, from Fluka Chemie AG, Switzerland), Tween 20 and the like. The amount of the analyte-releasing reagent employed is about 0.01 to 2% by weight and the reagent is applied in a manner similar to that described above for the stabilization reagent.

As mentioned above, the sample may be analyzed by any convenient method. Assays include, by way of illustration and not limitation, agglutination assays, precipitation assays, nephelometric assays, turbidimetric assays, immunoassays, coagulation assays, and so forth. The assays may involve members of a specific binding pair such as antigens, antibodies, receptors, and so forth. Such assays include immunoassays, receptor binding assays, coagulation assays, agglutination assays and the like. Detection of an assay result depends on the signal producing system chosen, example of which are set forth above. For example, where the label is a fluorescent label, signal is detected with a fluorometer, and so forth. For enzyme labels, the signal is often detected spectrophotometrically. Exemplary of assays employing enzyme labels are the EMIT® assay described in U.S. Pat. No. 3,817,837, the disclosure of which is incorporated herein by reference, the CEDIA assay, and so forth.

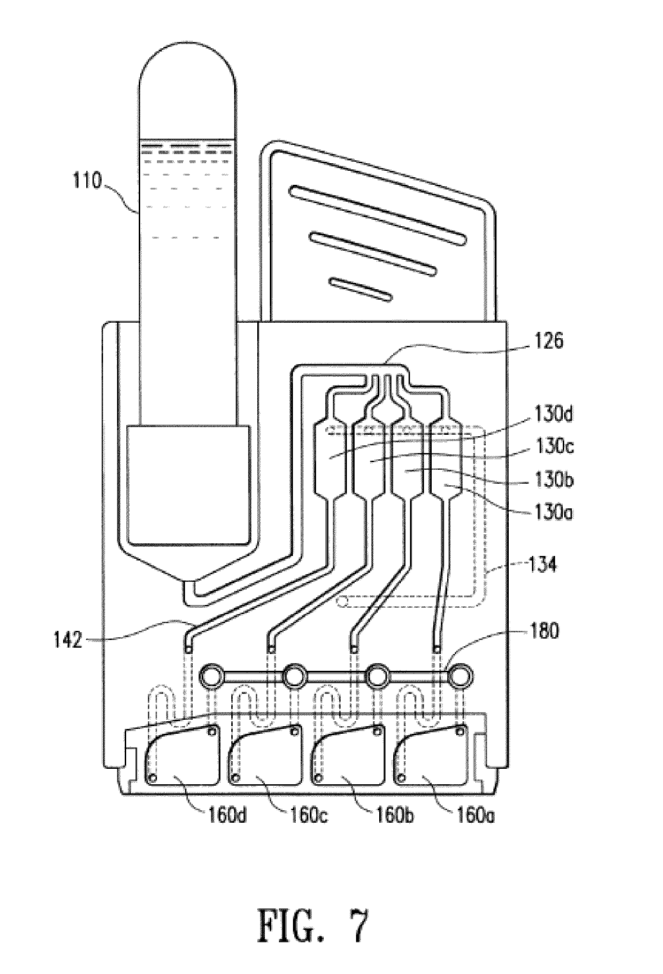

While the embodiments of FIGS. 1 and 5 have been illustrated having four and three second chambers, respectively, there is no inherent limitation upon the number of chambers, which usually will be between one and four. In addition, there may be more than one first chamber (see FIG. 7 depicting first chambers 130a, 30b, 30c and 30d) usually connected in series in fluid communication through channels or capillaries in a manner similar to that described for the first and second chambers. Generally, the last of the first chambers is in direct fluid communication with the first manifold. Likewise, the illustrated embodiments are equipped with magnetic mixing means, which may be substituted as desired with alternative motive means. It is contemplated that the observation of the sample in the assessment chamber will be by optical means, principally via fluorescence or infrared absorption.

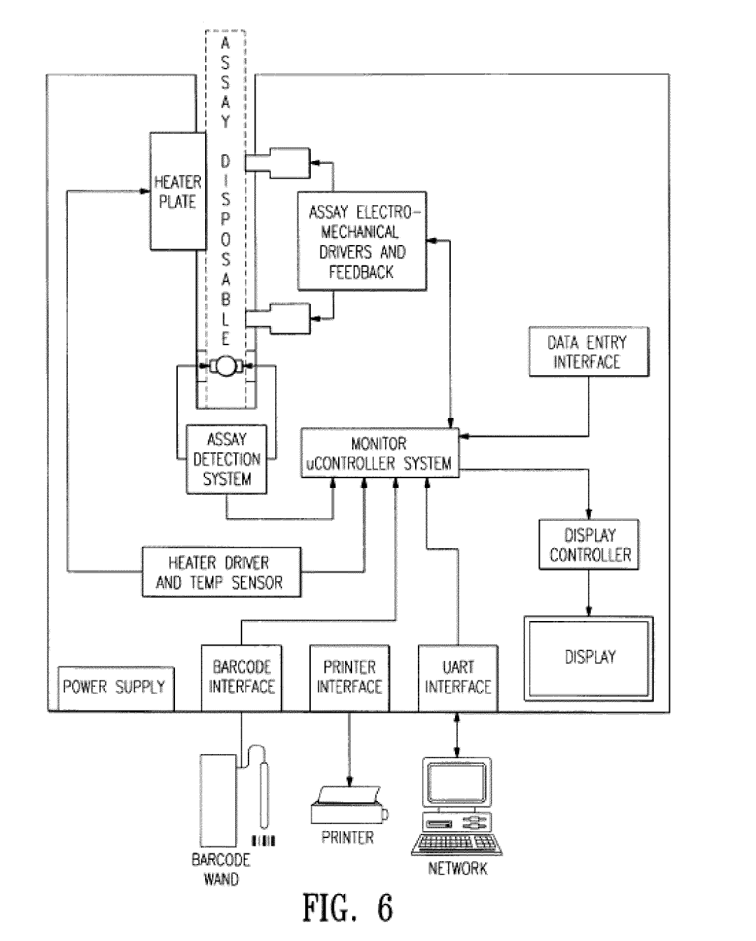

An example, by way of illustration and not limitation, of an instrument into which the present device may be employed is depicted in FIG. 6. The instrument includes a turbidimetric-based optical detection system that measures aggregation as an increase in light transmittance. Due to the ratio of bead size to the measurement wavelength, the light scattering is primarily forward (Mie) scatter. As a result, the chambers of the present device are illuminated by a narrow bandwidth emitter with detectors, mounted in direct opposition, to collect the in-coming light. The optical detector converts the light into an electrical current that is input into a transimpedance amplifier and converted to a voltage, which is the measured signal. This instrument is AC powered and is based on an embedded PC architecture. The instrument controls the assay sequencing, establishes and maintains the assay temperature, controls the reagent-sample mixing for the required duration, determines the result, displays result and status information to the user, and performs self-diagnostics. The instrument supports bar code data entry, printing of teat results to an external printer, and an RS-485 interface to interconnect to a laboratory network. The instrument has four independent optical detection channels comprised of narrow band emitters and high gain broadband detectors. Each detector output is A/.D converted at a rate of up to 16 Hz. The assay mixing is controlled by a solenoid or programmable clock-driven stepper motor that provides uniform mixing across all four channels. The temperature of the sample is controlled by a closed-loop feedback design utilizing a precision thermistor and a resistive heater element.

The present invention may be advantageously utilized in whole blood assays. For example, suitable platelet binding function assays which may advantageously utilize the method and/or device of the present invention are described in U.S. Pat. No. 5,854,005 (the '005 patent) and U.S. Pat. No. 6,016,712 (the '712 patent), the disclosures of which are incorporated by reference herein.

In the '005 patent, a 70 ml sample of blood plus anticoagulant is added to a borosilicate tube containing a buffer with 0.05 mM calcium chloride, a blue bead suspension (20 ml fibrinogen coated beads, 3 mm), and an activating peptide [5 10 ml (iso S)FLLRN NH2, 2 mM final concentration]. After the tube is capped and mixed, the blood is rocked on an end to-end tube mixer and viewed for the presence or absence of bead agglutination. The agglutinated beads are readily seen in the stream of blood as the tube is tilted back and forth, and the extent of agglutination is rated from O+ (no agglutination) to 4+ (extensive agglutination). The assay conditions were designed to yield an end point at 120 seconds in order to satisfy the practical needs for a rapid determination desired in a clinical setting. This method, while entirely satisfactory for its intended use, is however subjective and therefore operator dependent due to the method of assessing and reporting the results. Further, it is desirable to have the ability to generate a permanent, quantified record of various platelet functions.

The assay described in the '712 patent is based on the principle that fibrinogen coated microparticles exhibit a visible agglutination reaction in whole blood in the presence of activated platelets with normal GPIIb/IIIa receptors. Blockade of the GPIIb/IIIa sites by c7E3 antibody or other agents can be detected by inhibition of microbead agglutination. The assay, unlike other activated coagulation assays, is only minimally influenced by the anticoagulant effect of heparin and is believed to primarily reflect GPIIb/IIIa status, unless there is severe thrombocytopenia or serious qualitative platelet dysfunction. The presence of normal plasma levels of fibrinogen (2-4 mg/ml) also does not greatly influence the assay because of preferential interaction of the platelets with the immobilized fibrinogen. In practice, the assay requires the presence of an agglutination medium, preferably GPIIb/IIIa receptor land coated microparticles, a platelet activating agent, means for observing the aggregation of the microparticles, and means for recording, compiling, and displaying the results. Each of these is discussed more fully below.

Receptor Ligands:

A GPIIb/IIIa receptor ligand is a small organic molecule, polypeptide, protein, monoclonal antibody or nucleic acid that binds, complexes or interacts with GPIIb/IIIa receptors on the platelet surface. Platelet mediated aggregation of the microparticles results when the GPIIb/IIIa receptors on the surface of platelets bind, complex or otherwise interact with the GPIIb/IIIa receptor ligands on the particles or beads. Typical GPIIb/IIIa ligands include fibrinogen, monoclonal antibody 0E5 (Coller, et al., J. Clin. Invest. 72:325 (1983)), monoclonal antibody c7E3 (The EPIC Investigators, N.E. Journal of Med., 330:956 (1994)), von Willebrand factor, fibronectin, vitronectin and other ligands that have an arginine glycine-aspartic acid (RGD) sequence or other peptides or peptidomimetics that mimic this sequence (Cook, et al., Drugs of the Future 19:135 (1994)). RGD functionally equivalent ligands include gamma chain peptides, peptidomimetics and cyclic peptides with activity about the same as an RGD ligand surface through a suitable spacer. Examples of suitable ligands are disclosed in Beer, et al., Blood 79:117 (1992). Suitable GPIIb/IIIa receptor ligands include the peptide (glycine)n arginine glycine aspartic acid, wherein n is an integer from 220. The polyglycine portion of the ligand serves as a spacer and is covalently bound to the surface of the polymeric bead via the N terminal amino group. While the RGD sequence may participate in the binding of platelets, a gamma chain sequence forming a molecular mimic of the RGD sequence may be more important in the binding of fibrinogen to platelets (Coller, Platelet Morphology, Biochemistry, and Function, 1175). Optionally, an additional amino acid or oligopeptide that does not significantly interfere with the binding of arginine glycine aspartic acid to the GPIIb/IIIa receptor may be bound to the C terminus of aspartic acid by means of a peptide bond. In one embodiment, the GPIIb/IIIa receptor ligand comprises (Glycine)9-11-arginine glycine aspartic acid phenylalanine. Alternatively, the spacer portion of the ligand can comprise any moiety which causes the arginine glycine aspartic acid sequence to extend out from the surface of the microparticle sufficiently to allow binding between the ligand and GPIIb/IIIa receptors on the surface of platelets and does not significantly interfere with the ability of arginine glycine aspartic acid to bind with GPIIb/IIIa. Examples of suitable moieties include alkyl groups and polyglycol groups.

Activating Agents:

The thrombin receptor is a transmembrane protein that is present in platelets (Vu, et al., Cell 64:1057 (1992)). A thrombin receptor activator is a peptide, protein, antibody or small organic molecule that induces platelet activation via the thrombin receptor, i.e., which increases the rate of agglutination when platelets whose GPIIb/IIIa receptors are not blocked when the platelets are combined with a GPIIb/IIIa receptor ligand bound to solid surfaces. A suitable peptide is any peptide of appropriate sequence and size to activate platelets, as described above. The peptide can comprise thrombin, or a portion thereof, such that the amino acid sequence of the peptide or peptide mimic result in activation of the platelets. Vu, et al., identified the amino acid sequence of the thrombin receptor and proposed a mechanism of thrombin receptor activation. Thrombin cleaves the thrombin receptor protein, releasing a short receptor fragment and leaving a new amino terminal peptide on the platelet surface. The new amino terminal peptide activates the receptor by functioning as a tethered ligand that interacts with another region of the receptor to induce activation signals. A fourteen amino acid peptide (T14) corresponding to the new N terminus of the cleaved receptor protein is capable of aggregating platelets directly without prior thrombin cleavage (Vu, et al). However, the entire peptide is not required for activity because an eleven amino acid peptide (T11) lacking the three C terminal amino acids of T14 is twice as potent as T14 (see Coller, et al., Biochemistry 31:11713 (1992), the contents of which are hereby incorporated by reference). A peptide comprising the first five or six amino acids has also been shown to be active. (Vassallo, et al., J. Biol. Chem. 267:6081 (1992), Hui, et al., Biochem. Biophys. Res Commun. 184:790 (1992), Sabo, et al., Biochem. Biophys. Res. Commun. 188:604 (1992) and Scarborough, et al., J. Biol. Chem. 267:13146(1992).

The N-terminal serine group of the thrombin receptor activating peptides is essential to their ability to induce platelet aggregation,. This conclusion is based on the observation that acetylation of the N terminal serine of T11 results in loss of aggregating ability. In addition, T11 and T14 lose their ability to accelerate aggregation when incubated in plasma because the plasma component aminopeptidase M cleaves the N terminal amino acid. The presence of aminopeptidase M in whole blood can result in variability in the amount of time required for agglutination of the beads in the assay. Acetylation of the N terminus of the thrombin receptor peptide ligand is the traditional method for producing a peptide that resists cleavage by aminopeptidase M, but acetylation of this ligand eliminates receptor activator activity.

Other platelet activators can be used in place of the thrombin receptor activating peptides described above. For example, adenosine diphosphate (ADP), collagen, ristocetin, botrocetin, epinephrine, arachidonic acid and its metabolites including thromboxane A2, platelet activating factor, plasmin, serotonin, vasopressin, tissue plasminogen activator, streptokinase and immune complexes can be added, alone or in combination with other platelet activators, to increase the rate of agglutination of the beads in the assay of the present invention. Additionally, application of high levels of shear stress, and artificial surfaces such as those used clinically for prosthetic materials can also activate platelets (Coller, Platelet Morphology, Biochemistry, and Function, 1185).

Agglutination Media:

The agglutination media may be any suitable solid surface bearing a receptor ligand. Preferably the surface is a small polymeric bead or microparticle to which a GPIIb/IIIa receptor ligand is covalently bound or absorbed. The polymeric microparticles can be virtually any shape, but are generally spherical with uniform diameters ranging from about 0.1 mm to about 50 mm in diameter. Typical diameters are from about 1 mm to about 10 mm in diameter, most preferably about 6 mm. The composition of the particle may be any convenient composition, such as bioglas, organic polymers, e.g. polyacrylonitrile, polystyrene, polycarbonate, polymethacrylate, combinations thereof, or the like, or other material which absorbs in the infrared or can be made to do so with infrared absorbing dyes. For the most part the particle composition without the dye will not absorb significantly in the infrared region of interest, usually absorbing less than about 25% of the total light absorbed in that region compared to the particle doped with the infrared absorbing dye. Also, there will be many regions in the visual region in which the particle composition will be substantially transparent, as distinguished from carbon or colloid particles which do not transmit light over the visual and infrared region. Usually, at least 50 weight %, preferably at least about 75 weight %, will be of a size or diameter within the range indicated. The particles may be modified in a variety of ways. The particles may be chemically activated by having functional groups present on the surface of the particles, or be coated with a compound, e.g. protein, which may serve to substantially irreversibly (under the conditions of the processing and assay) bind to the dye. The coating compound may be the binding component, which will be involved in the aggregation of the particles, or other compound, usually being a protein. Alternatively, depending on the nature of the particles, the particles may not have chemically active groups, but rather provide binding by adsorption. In addition, infrared absorbing dyes which are stable under the conditions of formation of the particles, e.g. extrusion, may be mixed with the polymer prior to particle formation and the particle formed with the dye distributed throughout the particle.

The particles are loaded with a dye which absorbs in the infrared. Various dyes have been reported as useful in this absorption range. See, for example, Fabian, et al., Chem. Rev. (1992) 92:1197 1226. These dyes include bacteriochlorin, bacteriochlorophytin, meropolymethine dyes, benzoannulenes, vinylogous porphyrins, polymethine dyes, cyanines and merocyanines, and the like. The particular dye which is selected is one of convenience, availability, stability, compatibility with the particle, and the like. Specific dyes of interest include dyes of the class of phthalocyanines, napthalocyanines, metaled napthalocyanine dyes, and modified natural bacterochlorines. Specific example dyes include IR 140, 1,1′ Diethyl 4,4′ dicarbocyanine iodide, 1,1′ Diethyl 2,2′ quinotncarbocyanine iodide, Vanadyl,10,17,24 tetra tert butyl 1, 8, 15, 22, 25 tetrakis(dimethylamino) 29H,31H phthalocyanine, [RA800 (from Exciton), ProJet 830NP (from Zeneca). These dyes may be incorporated directly into the particle itself, through polymerization or passive adsorption. Alternatively, the dyes may be linked to the bead in combination with the binding component, such that they do not leach from the surface. The dyes will adsorb light in the range of about 750 to 900 nm, particularly in the range of about 750 to 850 nm. For samples with high levels of red blood cells, the light will be at about 800 nm.+−0.10 nm, which is the isobestic point for oxyhemoglobin and reduced hemoglobin. The amount of the dye employed with the particles will vary with the extinction coefficient of the dye in the light range of interest, the required sensitivity of the assay, the size of the particles, the mode of binding of the dye to the particles, compatibility of the dye with the particle matrix, and the like. Usually, loading will be in the range of about 1 to 20 weight percent, more usually 5 to 15 weight percent.

For example, the polymeric microparticle may be polyacrylonitrile beads with N hydroxysuccinimide ester groups on their surface (e.g., Matrex 102 beads from Amicon Corporation) (Coller, Blood, 55:169 (1980)). The N hydroxysuccinimide ester groups allow coupling of the N terminus of a peptide, protein or monoclonal antibody to the surface of the bead. Alternatively, the microparticle can be carboxylated polystyrene beads (Polysciences Inc.). The surface carboxyl groups of this bead can be coupled to the N terminus of the protein, peptide or monoclonal antibody by means of a carbodiimide coupling. The beads may be colored to render the results of the agglutination reaction easier to interpret. In a preferred embodiment, the beads are adapted to absorb light in the infrared region.

To eliminate effects of red blood cell oxygenation, an IR source of wavelength of about 800 nm is preferred since oxyhemoglobin and deoxyhemoglobin have the same optical absorption coefficient at 805 nm. Assay dependence upon the variable state of red blood cell oxygenation is thereby eliminated. Further, the hemoglobin isobestic point at 805 nm has the lowest absorption coefficient between 300 nm and 1000 nm. This results in the widest possible differential between light absorption by the red blood cells versus that by the agglutination medium (beads).

The agglutination medium is selected to have high absorption at about 800 nm. The ratio between the agglutination medium absorption coefficient and whole blood absorption coefficient should preferably be greater than about 4:1 at 800 nm. The absorption ratio for a particular assay is a function of both the absorption coefficient of the agglutination medium and the concentration of the agglutination medium in the assay sample.

The IR absorbing particles typically have the following properties: absorption target around 800 to 810 nm; reasonably broad half widths (>75 nm) around the absorption peak; little or no fluorescence (high fluorescence requires the use of a high pass optical filter), and a molar extinction coefficient of about 30,000.

Categories of IR absorbing particles which may be used in the assay include: latex particles dyed with IR dyes, D1 dye sole, carbon black particles, and liposomes with entrapped IR absorbers. The particles are typically spherical beads about 6 mm in diameter.

Generally, an hydrophobic IR dye soluble in organic solvents is preferred. Latex particles are dyed with the IR dye according to established methods using dyes which absorb in the visible range at about 800 nm (see Lee Bangs, Uniform Latex Particles). Latex offers the advantage of size uniformity. Water insoluble dyes may be induced to form colloids (sols, sub micron or larger particles) when a solution of the dye in a water miscible solvent is added to water. Carbon black absorbs well in the IR region of the spectrum and can be dispersed in aqueous solutions by sonication. Liposomes with entrapped IR absorbers, e.g., a water soluble IR dye, can be tailored to the desired sizes and fibrinogen bound to the surface via an anchor compound, such as palmitoyl chloride.

Typically, the concentration of beads is adjusted so that the platelet/bead ratio is from about 1.9 to about 2.8. A GPIIb/IIIa ligand may be covalently or ionically coupled to the bead, or the ligand may be simply coated on the bead. The ligand bearing beads may be lyophilized. A representative formulation for lyophilization is about 10 mg/ml beads, 75 mg/ml fibrinogen, and 200 mg/ml bovine serum albumin.

The time for the assay will depend upon the manner in which the measurement is taken. Where zero time is carefully controlled, one may take one or two measurements at different time intervals to determine the absolute infrared transmission at the time intervals or determine the rate of formation of the aggregation. Alternatively, one may take a plurality of measurements over the time course of the assay and analyze the slope beginning at a fixed time from the time of mixing. The data may be analyzed by any convenient means, particularly using an algorithm which can manipulate the data in relation to calibrators and/or controls. The total time of the readings from the zero time (time of mixing), may range from about 10 sec. to 5 min., more usually about 30 sec. to 5 min., and most commonly about 30 sec to 2 min.

Usually, the result will be compared to a calibrator, which may be performed concomitantly or have been performed previously or may be provided as a standard curve. The calibrators will vary depending upon the nature of the component of interest. Samples having known amounts of the component of interest may be prepared and performed in the assay and the results charted so as to be able to translate the measurement obtained with the sample to the standard. In some instances controls will be used, where the base value may vary depending on the source of the sample. The particular control will be associated with the sample and the component of interest.

Where platelet aggregation is to be measured, because of interest in the platelet status of an individual, which may be the natural status or the status resulting from administration of a drug, the sample typically will be in effect whole blood, which has been subjected to less than about 50%, generally less than about 20% dilution.

The amount of beads should provide a ratio between the agglutination media absorption coefficient and whole blood absorption coefficient of greater than about 4:1 at 800 nm, generally, not more than about 10:1 at 800 nm. The optimal absorption ratio may be achieved by configuring both the light absorbing characteristics of the agglutination media and the concentration of the agglutination media in the assay sample.

Sample may be moved into the first chamber by adjusting the pressure in the device using the pneumatic circuit. Generally, the volume of the sample transferred into the first chamber is about 0.1 to 2 ml. Typically, the blood in the first chamber is heated to about 37° C. Then, the blood is moved into the second chambers as described above where it mixes with the particles and other reagents.

The mixture of citrates, whole blood, particles and activating agent typically is gently agitated by causing the mixing ball contained in the second chambers to magnetically activate. Mixing insures homogeneity and the mild agitation is continued so as to maintain homogeneity without impeding aggregation formation. The temperature for the medium will be maintained at a constant temperature. After a short time, generally under 30 sec., usually under about 10 sec., readings are begun by illuminating the sample with light at about 800 nm. The total time for the readings will generally be under about 5 min., usually 3 min. When one is determining the rate of change to determine the change in slope with time, the number of data points per second may range from about 0.01 to 100, more usually from about 1 to 50. Thus, one reagent may then be combined with the particles coated with fibrinogen in the same manner as the sample. If desired, the buffered medium may be augmented with blood constituents, such as red blood cells, serum albumin, immunoglobulins, or other significant constituent of blood, which does not participate in the aggregation of the particles. A convenient buffer medium is HEPES sodium chloride buffer comprising from 1-5 mg/ml protein, e.g., BSA.

In one aspect, the present invention is directed towards apparatus for controlled heating of a blood sample within an assay system or other system requiring similar heating requirements. In general, heating of whole blood (or other blood or biological fluids) in assay applications such as those described herein can be characterized by a heating profile defining a range of initial temperatures as well as target temperatures and associated response time(s). In such a heating profile, the blood is received from a sample of blood taken from a patient or from a stored blood sample collected previously from the patient, and then introduced to the assay system for analysis. The analysis typically must be performed at a predefined target temperature within a predefined time.

In an exemplary embodiment, the blood sample is collected in a test tube or other collection apparatus and then transferred to a disposable, one-time use cartridge having two or more chambers including a staging area and assay mixing well, with the cartridge configured to be inserted into or positioned in the assay system at the time of testing. The cartridge may be one of the sample containers or other sample receiving elements previously described herein or may be another type of sample collection and storage container.

After receiving the cartridge, the blood sample is rapidly heated to a predefined testing temperature, after which the assay is performed. In an exemplary heating profile, the blood is heated from an initial temperature of below 37 degrees Celsius to a target temperature of 37 degrees Celsius, within two minutes. Typical temperature tolerances are one to two degrees Celsius of the target temperature.

Heating of the blood sample may potentially be done in a number of ways, including by conductive or radiant heating of the cartridge and blood sample stored within the cartridge. However, it is very difficult to perform controlled heating of the blood sample within the constraints of the heating profile so as to normalize heating of the blood throughout the sample and prevent overheating of the sample in specific regions, such as at the surface of the sample. In addition, heating through use of a heating plate or fluid bath may take too long for proper sample processing. Moreover, heat distribution is a complex function of both the heating apparatus and the characteristics of the blood sample, and therefore may be difficult to analyze, particularly when optical heating is used. Accordingly, one aspect of the present invention relates to specific process steps and associated apparatus to perform such heating in a consistent, uniform fashion while preventing overheating of regions of the sample.

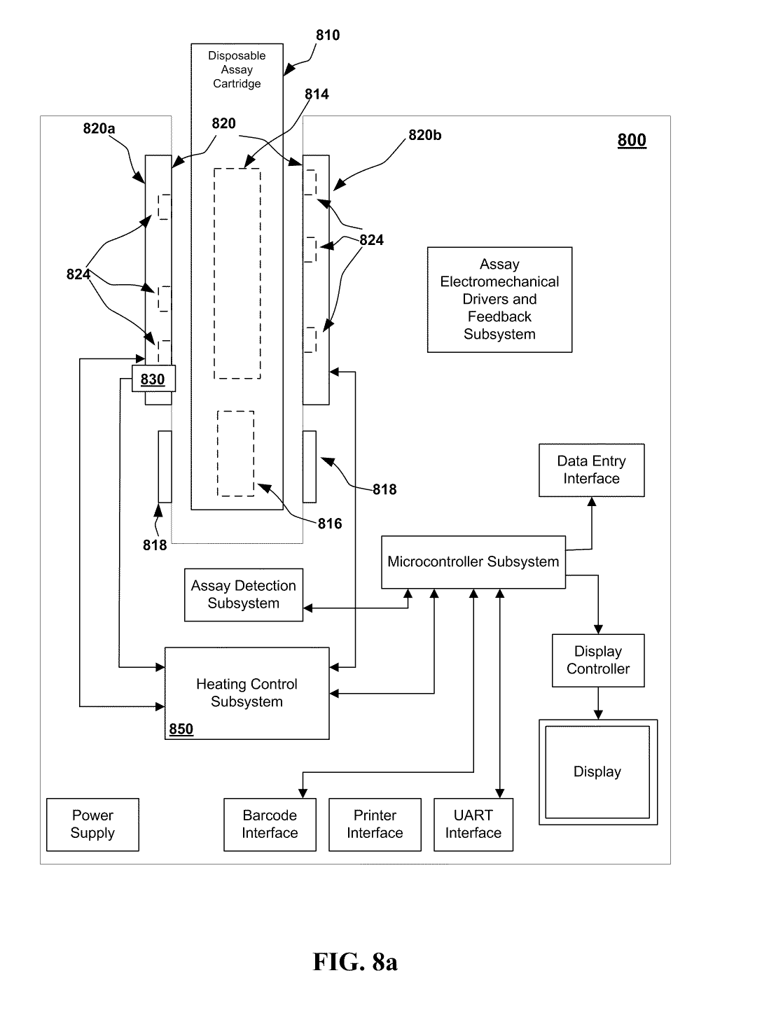

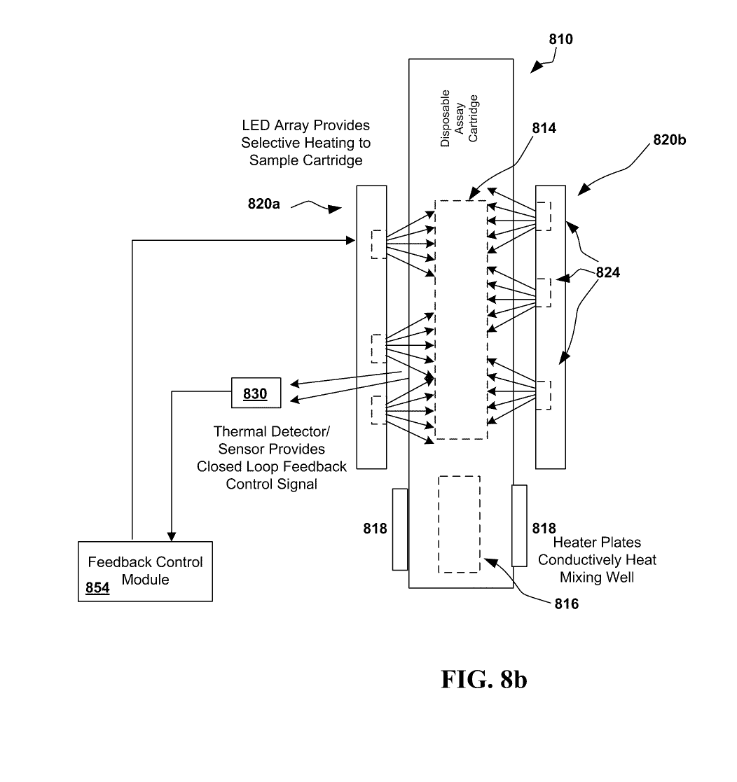

Attention is now directed to FIG. 8a which illustrates an embodiment of aspects of the present invention. System 800 is an assay instrument providing similar functionality to the system shown in FIG. 6; however, instrument 800 is configured to provide optical heating and sensing of the sample temperature rather than using a heater plate as shown in FIG. 6.

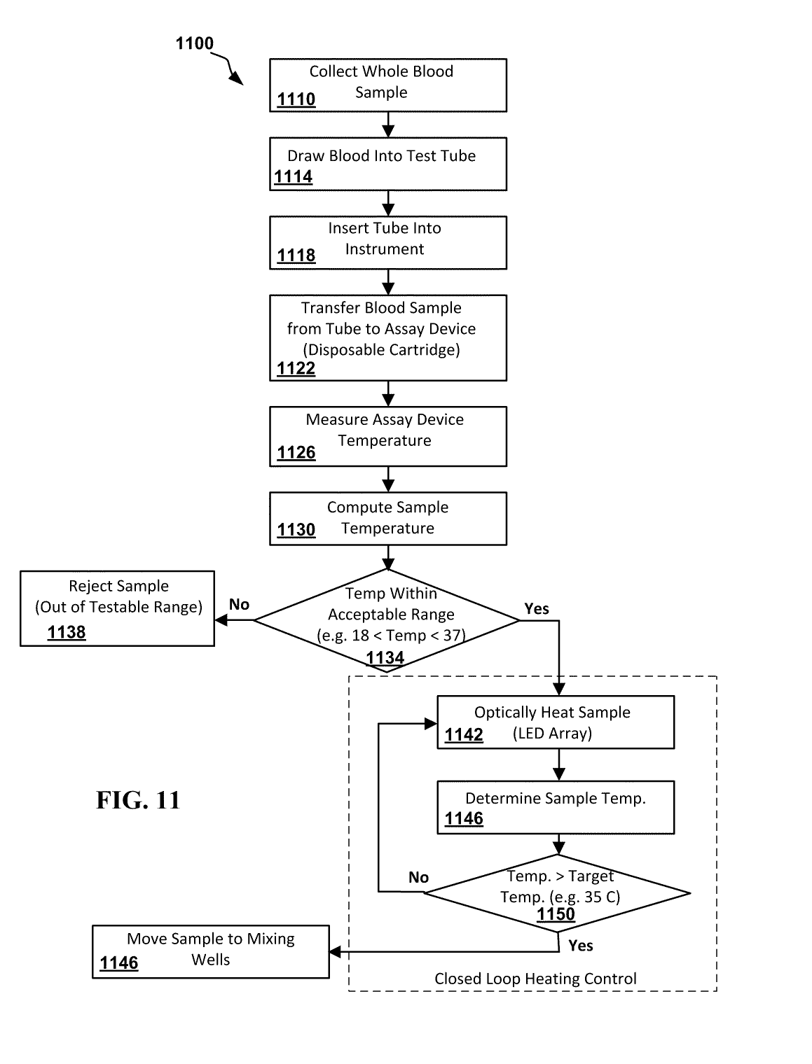

In operation, a blood sample, which is whole blood in an exemplary embodiment, is collected from a patient and typically stored for an initial time period prior to being transferred to disposable assay cartridge 810 (also described herein as a “cartridge” for brevity). The blood sample may be initially stored in a test tube (not shown) or other sample collection apparatus, and then transferred to an assay device or cartridge 800, such as is described elsewhere herein, with the cartridge configured to be inserted and/or housed in instrument 800. An exemplary process workflow 1100 describing this process is shown in FIG. 11 and described subsequently herein.

After transfer to the cartridge 810, the blood sample is positioned in a staging area or chamber 814 of the cartridge 810, where it is then heated in the assay system 800 prior to being transferred to an assay mixing chamber or well 816 for testing.

System 800 includes a housing assembly configured to receive the cartridge and position the cartridge adjacent to other instrument components including heating element(s) and a heating control subsystem. System 800 further includes a heating array, shown as a plurality of optical emitters disposed in an array element 820 in the embodiment of FIG. 8a, with the array element further divided into two sub-arrays 820a and 820b in the embodiment shown. In alternate embodiments, the heating array may comprise other configurations, types and numbers of array configurations and elements. In typical embodiments, the heating array is disposed in the housing element. As used herein, the term “array” means a combination of one or more elements (such as optical emitters or other elements for providing energy to facilitate heating of the sample) arranged in a systematic configuration. Array elements are collections of emitters comprising the array, which may be housed or otherwise mechanically disposed in the instrument. In an exemplary embodiment, an array element is a plurality of LEDs arranged on a printed circuit board (PCB) or other LED mechanical and/or electrical mounting structure. A heating array will typically include two or more emitter elements on an array element, with the emitters positioned on the array element adjacent to or in proximity to the sample so as to provide optical energy to optically heat the sample and/or the sample storage medium, such as a cartridge with an associated chamber or storage area. In typical embodiments, the heating array is configured to output approximately 1 to 3 watts of optical output; however, in other embodiments, smaller or larger output power levels may alternately be used. In addition, power level may be varied during the heating cycle in some embodiments.

In various embodiments, optical emitter array element 820 may be arranged in a variety of configurations depending on the specific shape and size of the disposable cartridge 810; however, in any of these configurations, optical emitter array element 820 is configured so as to provide optimal blood sample heating within the staging area in accordance with a selected heating profile. Heating profiles define requirements for blood heating in order to provide acceptable samples at specific temperatures. Accordingly, the heating profile may be defined by industry standards as well as by specific blood (or other biological fluid) properties or based on specific testing device properties or characteristics so that the sample is in an appropriate physical state at an appropriate temperature for testing.

As noted previously, an exemplary heating profile requires that the received blood sample be within a predefined input temperature range of 18 to 35 degrees Celsius, with the sample then being heated to a desired target temperature of 37 degrees Celsius within two minutes. Other profiles may alternately be used in various embodiments.

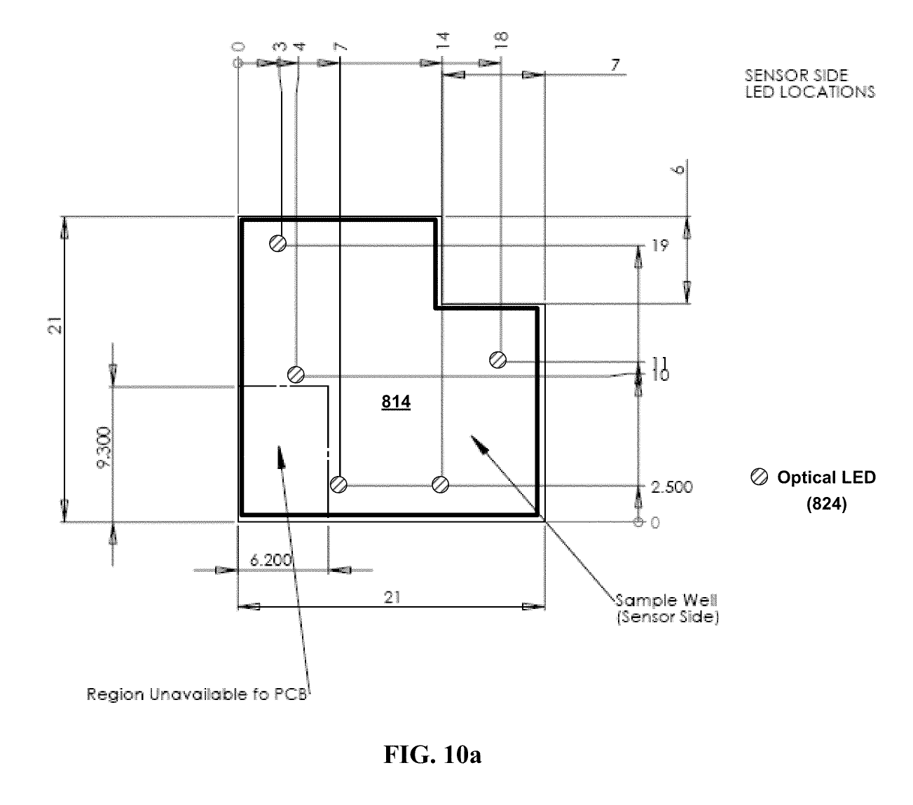

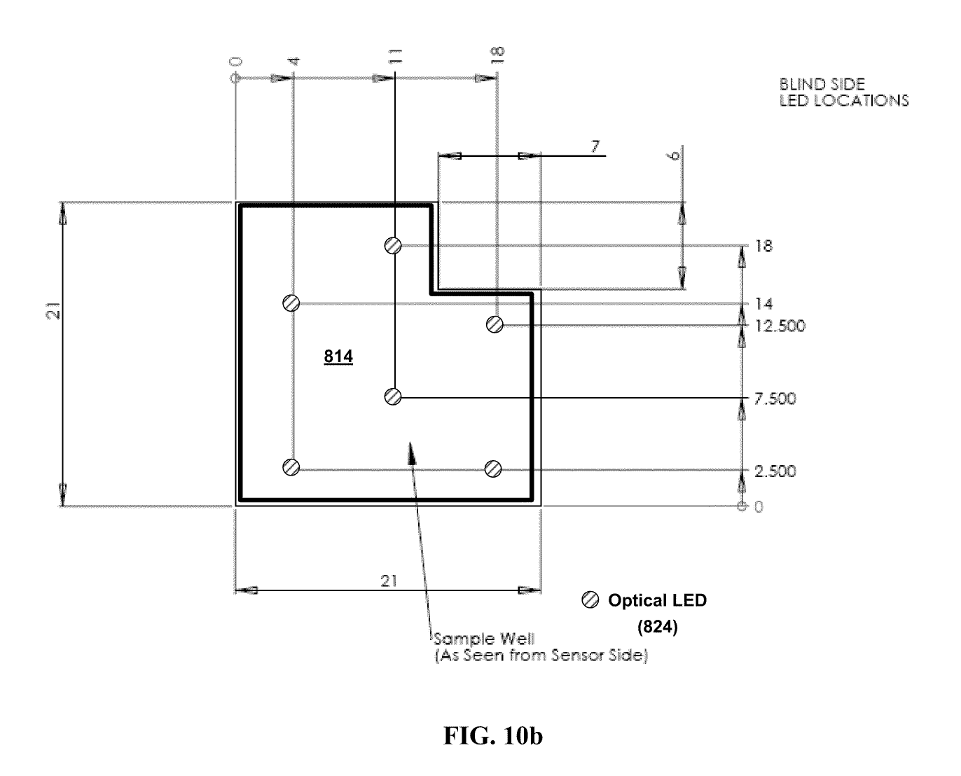

In the embodiment shown in FIG. 8a, which is based on a assay product that may be provided by Accumetrics, Inc., assignee of the present invention, the optical emitter array element 820 comprises a plurality of emitter elements 824 arranged on two emitter array sub-arrays 820a and 820b. Sub-arrays 820a and 820b are positioned within the instrument 800 adjacent to the cartridge 810 so as to optically illuminate opposite sides of the cartridge 810 and associated staging area 814. In this fashion, heating is distributed simultaneously from both sub-arrays 824a and 824b so as to more uniformly heat the blood sample within the staging area 814. FIGS. 10a and 10b illustrate an embodiment of an LED emitter array configuration that has been optimized for a specifically shown staging area as used in an Accumetrics product.

In an exemplary embodiment, the emitter elements 824 are high brightness light emitting diodes (LEDs), with an output power level, switching characteristics, and operating wavelength range selected so as to maximize emitter efficiency while providing uniform heating to the blood sample. Selection of specific LED devices (or other emitter elements) for the system 800 so as to provide uniform heating at a low cost is generally a difficult problem. While LEDs are available for operation over a wide range of wavelengths, selection of an appropriate emitter device is constrained by factors that include cost, size, power consumption, optical output power level, and other physical characteristics of the device, as well as the geometry and dimensions of the cartridge 810 and staging area 814. In particular, selection of an appropriate operating wavelength for the LEDs (or other emitters in some embodiments) is a non-trivial problem.

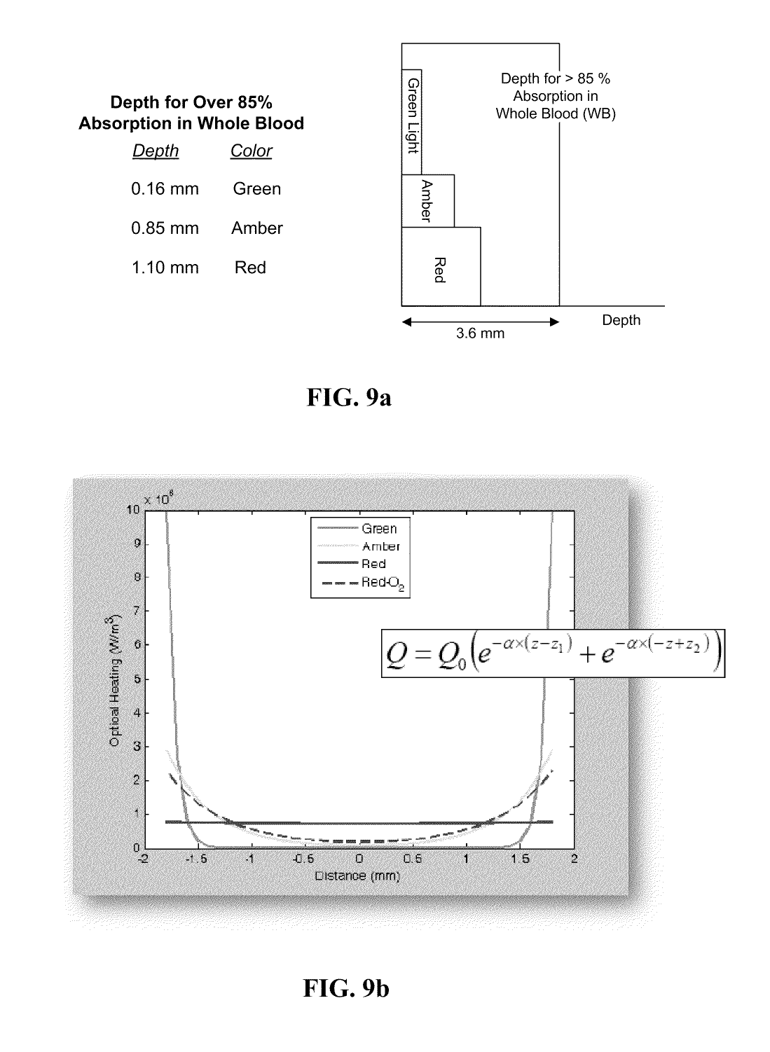

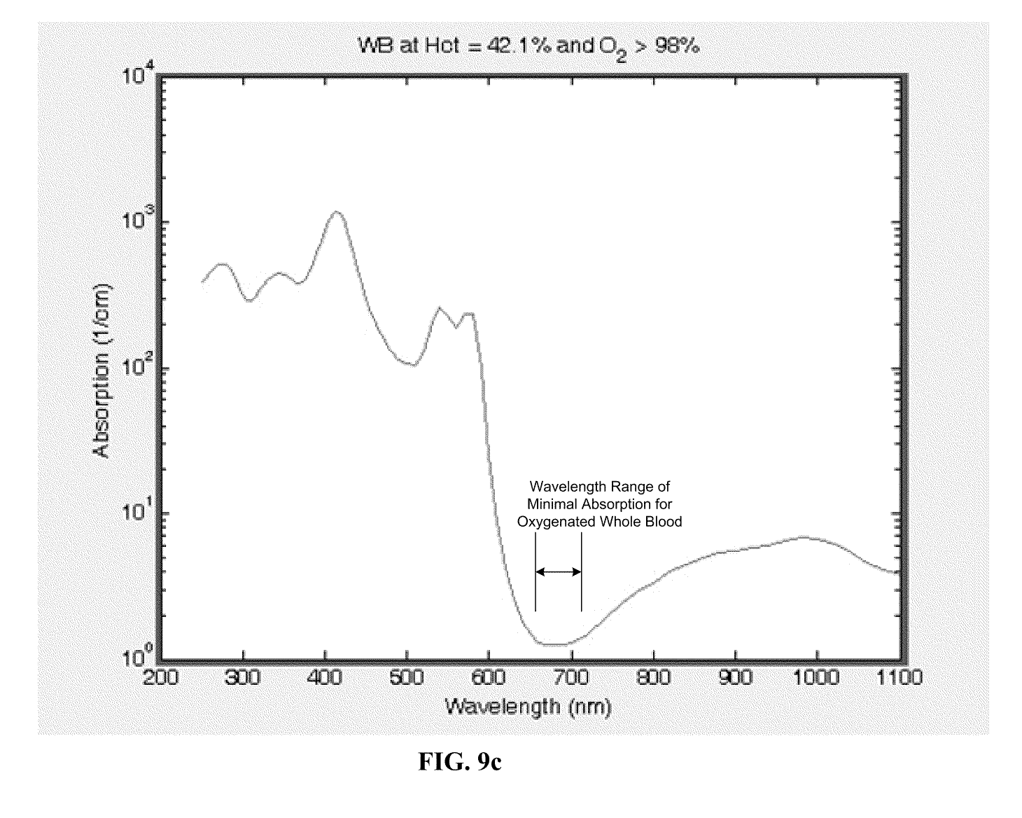

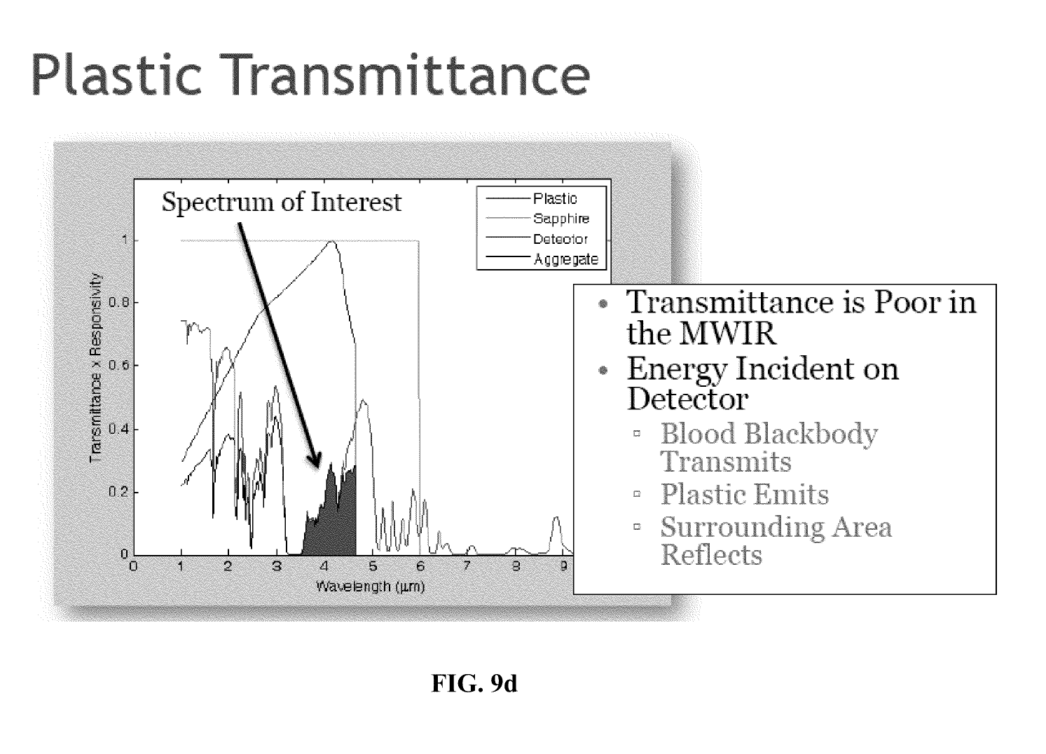

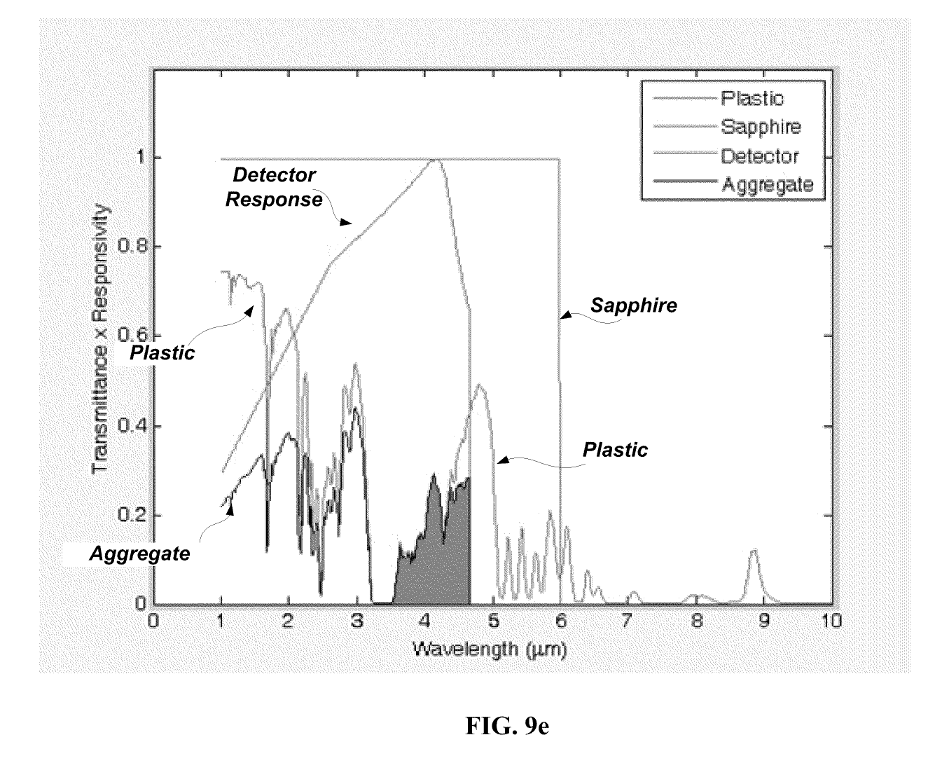

Intuitively, the absorption of optical energy by the blood sample (or other fluid under test) would be a primary characteristic of interest; however, selection of an operating wavelength or range of wavelengths based on maximizing absorption can lead to highly non-uniform heating of the blood sample. This is illustrated in FIG. 9a, which shows the depth of penetration for various colors of light in whole blood. Green light has the highest absorption and consequently the shortest depth, while red light penetrates nearly 10 times as far. Accordingly, selection of an operating wavelength or range of wavelengths is a function of the desired depth of penetration within the target fluid. If the depth of penetration is too shallow for the selected staging area and sample under test, significant surface overheating can occur, which prevents the goal of uniform sample heating.

FIG. 9b further illustrates this constraint. As shown in FIG. 9b, green light is substantially absorbed within 0.2 mm of the edge of the sample (shown in FIG. 9b as being illuminated simultaneously from opposite sides), whereas red light provides much more uniform absorption, and corresponding heating, throughout a 1-2 mm sample thickness. Consequently, selection of an emitter element operating in the red visible light region is desirable since it will avoid overheating of the edges for sample thicknesses on the order of 1-2 mm and provide distribution of the optical energy throughout the sample thickness.

In some embodiments, the emitter element will be configured to operate within a narrow band of wavelengths about a center wavelength (for example, in a band of wavelengths of 20-40 nanometers about a center wavelength of 630 nanometers). Alternately, the emitter elements may be configured to operate over a broader range of wavelengths (i.e., within the red wavelengths of visible light) and/or over a set of different wavelength ranges (i.e., within red and green wavelengths, blue and yellow wavelengths, etc.). Various combinations of emitters and arrangement of emitters elements in arrays are possible based on a specific target sample and desired heating profile. In addition, other types of emitter devices, in addition to LEDs, may be used in some embodiments. These may include, for example, tungsten emitters, laser devices, or other devices capable of generating energy in optical wavelengths.

In addition, in some embodiments the heating array may comprise two or more types of heating elements, with the different types of elements configured to provide heating at different wavelengths or via different heating mechanisms. For example, in some embodiments, optical energy may be provided at both narrowband wavelengths for targeted heating as well as via broader wavelengths or ranges of wavelengths. In addition, in some embodiments, optical heating may be combined with conductive and/or radiant heating, such as via contact heating, thermal pool heating, radiant (i.e., IR) heating, or other combinations of heating mechanisms.

Attention is now directed to FIG. 9c, which illustrates a model of absorption of oxygenated whole blood as a function of wavelength. This model is from M. Meinke et al., “Empirical model functions to calculate hematocrit-dependentoptical properties of human blood,” Applied Optics, V46, N10, April 2007, which is incorporated by reference herein in its entirety. The wavelength range of minimal absorption shown in FIG. 9c provides a potential advantageous operating range if minimal absorption in the targeted sample (for example, for oxygenated whole blood) is a desired characteristic. It is noted that the curve of FIG. 9c will generally be different for different fluids, as well as for different oxygenation levels, and therefore the optimal wavelength may vary for other fluids or for whole blood having different oxygenation levels. Consequently, various combinations of emitter elements may be used to target heating based on the specific sample material composition and absorption characteristics, as well as based on characteristics of materials used in the the assay instrument or associated cartridges.

Returning to FIG. 8a, instrument 800 also includes a heat sensor 830, and a heating control subsystem 850. The heat sensor 830 is used to determine the temperature of the blood sample based on heating provided by the array element 820, and the temperature information is then provided to heating control subsystem 850 where closed-loop control is implemented (i.e., the sensor information is used to provide a driving signal to the LED emitters 824 to attain the target temperature). After the sample is heated to the desired target temperature, the sample is then transferred to the mixing well 816 for the assay process.

FIG. 8b illustrates additional details of an embodiment of instrument 800 and the associated disposable cartridge 810. As shown in FIG. 8b, the emitter elements 824 of array element 820 provide, at a selected wavelength, optical radiation to the cartridge and blood sample stored within the cartridge in the staging area 814 so as to uniformly heat the sample to the target temperature. A feedback control module 854, which may be included in the heating control subsystem 850, implements closed loop control of the heating process based on a sensed temperature of the sample or associated cartridge by selectively controlling the output of the emitters 824. This may be done by a variety of methods including pulse-width modulation (PWM) control of the duty cycle of the emitters 824 or amplitude control of the output level of the emitters.

In general, the configuration of the LED emitters 824 within the array 820 will be non-symmetric (as is shown, for example, in FIGS. 8a and 8b as well as FIGS. 10a and 10b) in order to account for the shape and dimensional characteristics of the array element 820, the cartridge and staging area shapes and dimensions, as well as the characteristics of the sample material (i.e., blood or other fluids) under test. Details of a process for determining an optimized emitter element configuration are further described subsequently herein in FIG. 10c.

In an exemplary embodiment, the emitters 824 may be a hi-output red LED device such as the Luxeon LXML-PD01-0030 or LXML-PD01-0040 devices. These devices provide optical output at 620-640 nanometers, with a typical luminous flux (lm) or radiometric power (mW) of 65-85 lm.

As the sample is heated by the optical emitters 824, a sensor element 830 is used to determine the temperature of the sample both initially and during the heating process to facilitate closed-loop control of the heating process in conformance with the desired heating profile. While various types of thermal sensors might be used, in an exemplary embodiment, a non-contact thermopile device may be used so as to provide optical sensing of the temperature based on conversion of thermal energy to electrical energy. In some embodiments, the sensor 830 may be a thermopile sensor device. Suitable sensors may include sensors provided by Dextor and Melexis (low cost sensors) as well as Cal Sensors (higher performance sensors), or others. Low cost thermopile devices detect the temperature using a wavelength of 5-14 micrometers. This enables the measurement of the temperature of the assay device from which the temperature of the sample can be inferred. Higher cost PbSe sensors measure using a wavelength of 3-5 micrometers enabling direct measurement of the temperature of the sample. Use of the PbSe technology requires the use of large bias voltages and cooling to achieve the necessary performance. In addition, the sensor 830 will generally compete with the optical emitters 824 for space (i.e. mechanical placement) within the instrument, which may make smaller sized sensors more desirable in some applications where space is limited.

In typical implementations, use of both non-contact optical heating and non-contact optical sensing may provide advantages including the ability to quickly raise the temperature of the sample while also providing uniform sample heating, fast response times for the optical output so as to preclude overheating, as well as other potential advantages.

In an exemplary embodiment, the sensor element 830 detects the temperature of the cartridge material adjacent to the blood sample as a proxy for the sample temperature. This approach may provide advantages in cost and implementation. In particular, the emission of blackbody radiation, indicating the temperature of the sample occurs at wavelengths of 8-12 micrometers. As such, use of the cartridge material to infer the sample temperature is desirable since optical measurements of the sample temperature may not be practical if the optical absorption of the cartridge material is high (i.e., the optical emission from the sample at the sensor operating wavelength is absorbed by the cartridge material). In order to optimize both heating and sensing performance, the cartridge material is preferably selected based on its ability to be transparent to light at the optical heating emitter output wavelength so as to minimize direct heating of the cartridge by the optical illumination. The cartridge material should also be transparent to the wavelength used to measure the assay results. In addition, the cartridge material and geometry are preferably selected so at to be able to rapidly conduct heat from the sample so that the cartridge temperature closely matches the sample temperature. This may facilitate performance and speed in sensing temperature at sensor element 830. FIGS. 9d and 9E illustrate transmittance characteristics for various materials including the cartridge material (plastic), the sensor window (sapphire), the PbSe sensor response characteristics (detector), an the product of all of these (aggregate). The region labeled “Spectrum of Interest” indicates the small portion of the infrared spectrum which can be used to sense the sample temperature directly using the PbSe sensor. This illustrates the challenges in sensing temperature of the sample directly.

The feedback control module 854 is configured to implement closed-loop feedback control of the heating process. This may be done by control implementations as are known in the art such as by bang-bang control (i.e., on/off, sensing control), by pulse width modulation (i.e., where the output level of the optical emitter elements 824 are controlled by the duty cycle or pulse width of a controlling signal), and/or by amplitude control of the emitter elements, or by other control system methods. In any of these approaches, the control module implements heating control of the emitters such that the blood sample is uniformly heated from an initial temperature within a predefined target temperature range, to the target temperature, within a predefined time period. This is typically done primarily using optical heating; however, optical heating may be combined in some embodiments with other heating mechanisms, such as conductive or radiant heating.

After the sample is heated to the target temperature in the staging area 810, it is transferred to the mixing well 816 for the assay testing. Instrument 800 may include heater plates 818 as shown in FIGS. 8a and 8b to provide conductive heating to the mixing well 816 while performing the assay process.

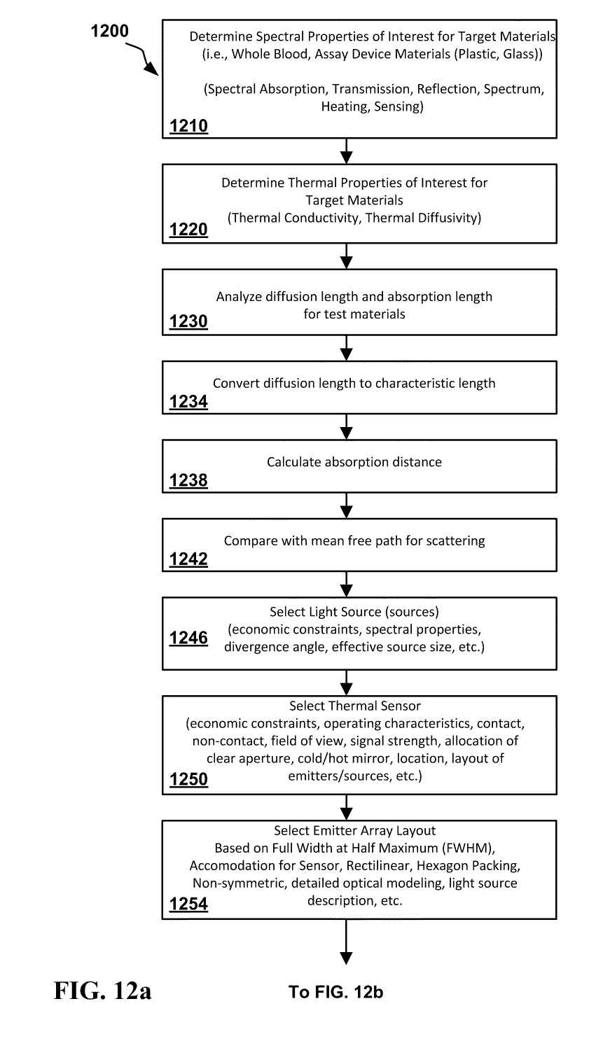

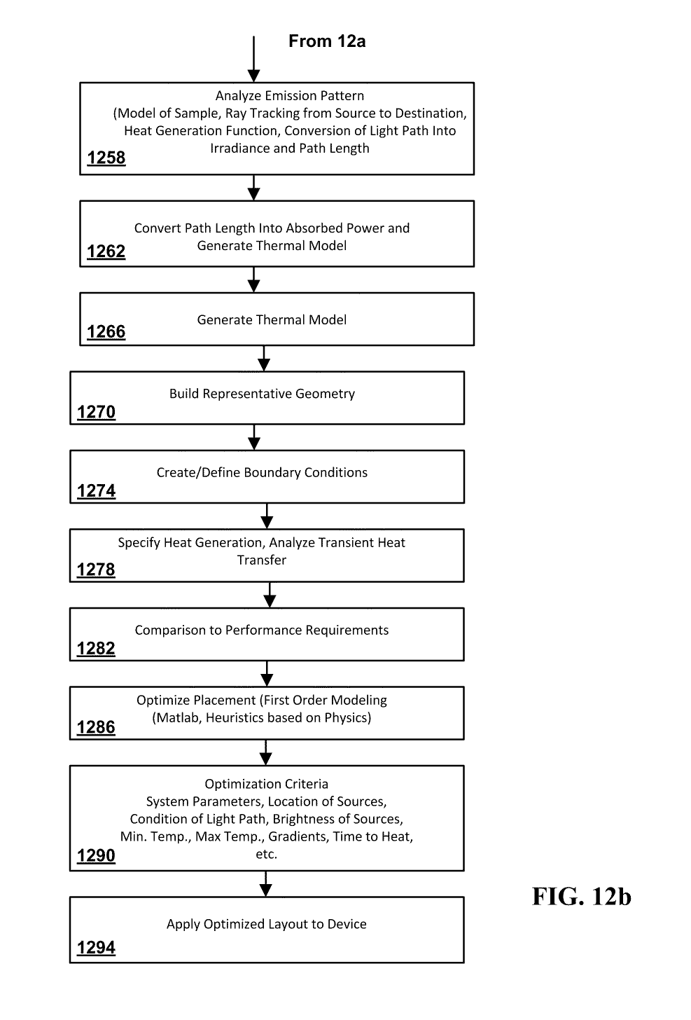

Attention is now directed to FIG. 12 which illustrates a process workflow for selecting an emitter element 824 and associated emitter configuration. In general, this selection process relies upon numerical methods, making it difficult predict the outcome a priori. The resulting emitter element pattern (within the array 820) is non-symmetrical, as shown by the embodiment of FIGS. 10a and 10b, which are an optimized configuration for a specific Accumetrics product.

FIGS. 10a and 10b illustrate an exemplary embodiment of an array element 820 layout, where FIG. 10a illustrates one side or sub-array 820a while FIG. 10b illustrates the other side or sub-array 820b. The sub-array shown in FIG. 10a is the side having the thermal sensor 830. This configuration was determined through a series of steps as described by the process shown in FIG. 12, based on specific LED elements and the cartridge and staging area configuration for a product provided by Accumetrics. It is noted that other configurations will need to be determined for different cartridges and associated staging areas, such as by using the proces shown in FIG. 12.

Attention is now directed to FIG. 11 which illustrates an embodiment of a workflow associated with the present invention. In this workflow, a whole blood sample is collected at stage 1110, with the blood then drawn into a test tube or other blood storage apparatus at stage 1114. The tube is then inserted or positioned in the assay testing instrument, such as instrument 800 of FIG. 8a, and the sample is transferred to a disposable assay device or cartridge at stage 1122. The initial or ambient temperature is measured at stage 1126 and a sample temperature is computer from this measurement at stage 1130. In particular, stage 1130 may include the stages of measuring the assay device between steps 1110 and 1114, then computing the temperature of the sample using the results from both steps 1126 and the measurement between steps 1110 and 1114. At decision stage 1134, if the sample temperature is greater than a maximum acceptable value (for example, 37 degrees C. in an exemplary embodiment), or less than a minimum temperature (18 degrees C. in an exemplary embodiment), the sample is rejected and the testing process terminated.

Alternately, if the sample is within an acceptable initial temperature range, it is then heated with either a timed heating cycle based on the sample material and initial temperature or (preferably) with closed loop control at stage 1142 to stage 1150. During each cycle, the temperature is sampled, and if it exceeds a target temperature (for example, 35 degrees C.), the sample is moved to the mixing wells for completion of the assay. Alternately, if the target temperature has not yet been attained, the sample is heated and measured again. This process may be repeated as necessary until the sample reaches the target temperature. In one embodiment a sensor is chopped at a 1 kHz rate, with cycling at a range of 1 Hz (the chopping feature is necessary for the PbSe detector only—it is neither necessary nor recommended when using a thermopile). However, other timing may obviously be used in various embodiments.

As a demonstration of the ability to heat whole blood in an accurate temperature controlled fashion, a heating array disposed in an array element configured as shown in FIGS. 10a and 10b was fabricated using red high output LEDs operating at approximately 620 to 640 nanometers. The heating array included 10 LEDS, with 5 LED emitters in each sub-array so as to uniformly heat both sides of the whole blood sample under test. Each LED element produced approximately 100 mW of optical output, for a total output of approximately 1 watts. The LEDs were operated at full power at approximately 700 mA of drive current.

The following example is offered by way of illustration and not by way of limitation. LED power percentage is with respect to maximum current (700 mA). Hct is by volume of red blood cells, not weight (mass). Temperatures are in degrees Celsius unless indicated otherwise. The following preparations and examples illustrate the invention but are not intended to limit its scope.

### EXAMPLE 1

Table 1 illustrates test results using this configuration. In particular, samples having initial temperatures between 18 degrees Celsius and 37 degrees Celsius were tested, with all samples being heated to the target or final temperature of between 36 and 37 degrees Celsius. Temperatures were measured in the test apparatus using a fine wire thermocouple in a modified assay cartridge. Temperature was recorded as the heated blood flowed out of the device staging area (staging well). The recorded temperatures are an average of five determinations, with the results demonstrating that optical heating was effective both in elevating the sample to, and maintaining the sample at a target temperature of 37 degrees Celsius. As shown in Table 1, the illustrated embodiment is capable of heating any of the samples from their initial temperatures to the 37 degree Celsius target within two minutes when operating at full power. Moreover, except for the 18 degree Celsius sample, the samples attained the target temperature range at half power well within 2 minutes.

TABLE 1
Optical Heating of Whole Blood to a Target Temp. of 37 C.
Optical heating of whole blood.

| initial sample | optical heating, | final sample | time to final |
|---|---|---|---|
| temperature, ° C. | LED power level | temperature, ° C. | temperature, s |
| 18 | 50% | 36.1 | 130 |
| | 100% | 36.6 | 82 |
| 32 | 50% | 36.6 | 100 |
| | 100% | 36.6 | 60 |
| 37 | 50% | 36.2 | 75 |
| | 100% | 36.5 | 44 |

In addition to achieving the appropriate temperature, the system must also be able to maintain the blood characteristics during the heating process. Table 2 reports factors used to check the potential deleterious effects of optical heating on blood samples by running samples on a modified assay cartridge in a VerifyNow® system manufactured by Accumetrics. During the test, the cartridge was removed following completion of the optical heating phase and the sample was withdrawn from the staging well and subjected to an automated CBC analyzer. In comparison to unheated control samples, optical heating did not significantly alter platelet (Plt) or red blood cell (RBC) counts or hematocrit (Hct). These test results are shown below in Table 2.

TABLE 2
Evaluation of Optical Heating on Sample Blood Counts
Evaluation of optical heating and sample blood counts.

| donor | initial sample | test | CBC results | | | |
|---|---|---|---|---|---|---|
| id | temperature, ° C. | condition | | Plt, per μL | RBC, per μL | Hct, % |
| 1 | 18 | unheated | average: | 157,000 | 5,100,000 | 44.1 |
| | | | st. dev.: | 1,732 | 51,962 | 0.3 |
| | | heated | average: | 153,000 | 5,156,667 | 44.4 |
| | | | st. dev.: | 5,568 | 25,166 | 0.4 |
| 2 | 18 | unheated | average: | 170,000 | 4,276,667 | 37.5 |
| | | | st. dev.: | 3,464 | 61,101 | 0.5 |
| | | heated | average: | 169,333 | 4,340,000 | 38.1 |
| | | | st. dev.: | 1,528 | 0 | 0.1 |
| 3 | 32 | unheated | average: | 142,333 | 4,370,000 | 37.5 |
| | | | st. dev.: | 2,517 | 26,458 | 0.2 |
| | | heated | average: | 142,667 | 4,376,667 | 37.6 |
| | | | st. dev.: | 10,116 | 70,946 | 0.6 |
| 4 | 32 | unheated | average: | 219,667 | 4,023,333 | 35.8 |
| | | | st. dev.: | 5,774 | 30,551 | 0.4 |
| | | heated | average: | 223,333 | 4,040,000 | 36.0 |
| | | | st. dev.: | 2,082 | 55,678 | 0.6 |

In addition, whole blood samples were assayed in P2Y12 cartridges in a VerifyNow® instrument made by Accumetrics. During the assay the sample was heated from room temperature to 37 degrees C. Raw detector data was collected during the two minute reaction phase of the assay and plotted to evaluate the assay dynamics. In each case, the expected assay signal characteristics (i.e., detector voltage vs assay progress, in seconds) were measured and closely matched the characteristics of whole blood heated on one side with a conductive heated platen, as is used in previous version of the Accumetrics instruments.

It is noted that in various embodiments the present invention may relate to processes such as are described or illustrated herein and/or in the related applications. These processes are typically implemented in one or more modules comprising systems as described herein and/or in the related applications, and such modules may include computer software stored on a computer or processor readable medium including instructions configured to be executed by one or more processors to perform the described functions. It is further noted that, while the processes described and illustrated herein and/or in the related applications may include particular stages, it is apparent that other processes including fewer, more, or different stages than those described and shown are also within the spirit and scope of the present invention. Accordingly, the processes shown herein and in the related applications are provided for purposes of illustration, not limitation.

As noted, some embodiments of the present invention may include computer software and/or computer hardware/software combinations configured to implement one or more processes or functions associated with the present invention such as those described above and/or in the related applications. These embodiments may be in the form of modules implementing functionality in software and/or hardware software combinations. Embodiments may also take the form of a computer storage product with a computer-readable medium having computer code thereon for performing various computer-implemented operations, such as operations related to functionality as describe herein. The media and computer code may be those specially designed and constructed for the purposes of the present invention, or they may be of the kind well known and available to those having skill in the computer software arts, or they may be a combination of both.

Examples of computer-readable media within the spirit and scope of the present invention include, but are not limited to: magnetic media such as hard disks; optical media such as CD-ROMs, DVDs and holographic devices; magneto-optical media; and hardware devices that are specially configured to store and execute program code, such as programmable microcontrollers, application-specific integrated circuits (“ASICs”), programmable logic devices (“PLDs”) and ROM and RAM memory devices. Examples of computer code may include machine code, such as produced by a compiler, and files containing higher-level code that are executed by a computer using an interpreter. Computer code may be comprised of one or more modules executing a particular process or processes to provide useful results, and the modules may communicate with one another via means known in the art. For example, some embodiments of the invention may be implemented using assembly language, Java, C, C#, C++, or other programming languages and software development tools as are known in the art. Other embodiments of the invention may be implemented in hardwired circuitry in place of, or in combination with, machine-executable software instructions.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for heating a whole blood sample for use in an assay, comprising irradiating said whole blood sample with monochromatic visible light of between 380 nm and 740 nm until the temperature of said whole blood sample is between about 35° C. and 40° C.

2. A method for heating a whole blood sample according to claim 1, wherein said monochromatic visible light is between 600 nm and 680 nm.

3. A method for heating a whole blood sample according to claim 1, wherein said monochromatic visible light is between 610 nm and 660 nm.

4. A method for heating a whole blood sample according to claim 1, wherein said monochromatic visible light is between 615 nm and 650 nm.

5. A method for heating a whole blood sample according to claim 1, wherein said monochromatic visible light is between 620 nm and 645 nm.

6. A method for heating a whole blood sample according to claim 1, wherein said irradiation from said monochromatic visible light raises the temperature of said whole blood sample to a temperature between about 36° C. and 38° C.

7. A method for heating a whole blood sample according to claim 1, wherein said irradiation from said monochromatic visible light raises the temperature of said whole blood sample to a temperature between 36.5° C. and 37.5° C.

8. A method for heating a whole blood sample according to claim 1, wherein said irradiation from said monochromatic visible light raises the temperature of said whole blood sample to a target temperature of about 37° C.

9. A method for heating a whole blood sample according to claim 1, wherein said irradiation is emitted by one or more light emitting diodes, incandescent filament light bulbs with filters, gas discharge lamps, fluorescent emitters, plasma emitters or lasers.

10. A method for heating a whole blood sample according to claim 9, wherein said irradiation is emitted by one or more light emitting diodes.

11. A method for heating a whole blood sample according to claim 10, wherein said one or more light emitting diodes are high output LEDs.

12. A method for heating a whole blood sample according to claim 10, wherein said irradiation is emitted by an array of LEDs.

13. A method for heating a whole blood sample according to claim 1, wherein the initial temperature of said whole blood sample is between about 18° C. and 34° C.

14. A method for heating a whole blood sample according to claim 1, wherein the initial temperature of said whole blood sample is between about 18° C. and 25° C.

15. A method for heating a whole blood sample according to claim 1, wherein the initial temperature of said whole blood sample is between about 21° C. and 25° C.

16. A method for heating a whole blood sample according to claim 1, wherein said temperature of said whole blood is raised to about 37° C. within 180 seconds.

17. A method for heating a whole blood sample according to claim 1, wherein said temperature of said whole blood is raised to about 37° C. within 120 seconds.

18. A method for heating a whole blood sample according to claim 1, wherein said temperature of said whole blood is raised to about 37° C. within 90 seconds.

19. A method for heating a whole blood sample according to claim 1, wherein said temperature of said whole blood is raised to about 37° C. within 60 seconds.

20. A method for heating a whole blood sample according to claim 1, wherein said temperature of said whole blood is raised to about 37° C. within 30 seconds.

21. A method for heating a whole blood sample according to claim 1, wherein said method further comprises determining the initial temperature of said whole blood sample.

22. A method for heating a whole blood sample according to claim 1, wherein said is configured to operate at an optical power output level of about 1-3 watts.

23. A method for heating a whole blood sample according to claim 1, wherein the temperature of said whole blood sample is determined using an optical sensor.

24. A method for heating a whole blood sample according to claim 1, wherein the thickness of the whole blood sample is between about 2 mm and 4 mm.

25. A method for heating a whole blood sample according to claim 24, wherein the thickness of the whole blood sample is about 3.6 mm.

26. A method for determining platelet binding function in a whole blood sample comprising irradiating said whole blood sample with monochromatic visible light of between 380 nm and 740 nm until the temperature of said whole blood sample is between about 35° C. and 40° C., combining said whole blood sample with an agglutinating system to form an assay mixture, irradiating said assay mixture with infrared light, determining the optical transmittance of said assay mixture to infrared light, wherein said optical transmittance is an indication of binding function of said platelets in said whole blood sample.

27. A method according to claim 26, wherein said monochromatic visible light is between 600 nm and 680 nm.

28. A method according to claim 26, wherein said monochromatic visible light is between 610 nm and 660 nm.

29. A method according to claim 26, wherein said monochromatic visible light is between 615 nm and 650 nm.

30. A method according to claim 26, wherein said monochromatic visible light is between 620 nm and 645 nm.

31. A method according to claim 26, wherein said irradiation from said monochromatic visible light raises the temperature of said whole blood sample to a temperature between about 36° C. and 38° C.

32. A method according to claim 26, wherein said irradiation from said monochromatic visible light raises the temperature of said whole blood sample to a temperature between 36.5° C. and 37.5° C.

33. A method according to claim 26, wherein said irradiation from said monochromatic visible light raises the temperature of said whole blood sample to a temperature of about 37° C.

34. A method according to claim 26, wherein said irradiation is emitted by one or more light emitting diodes, incandescent filament light bulbs with filters, gas discharge lamps, fluorescent emitters, plasma emitters or lasers.

35. A method according to claim 34, wherein said irradiation is emitted by one or more light emitting diodes.

36. A method according to claim 35, wherein said one or more light emitting diodes are high output LEDs.

37. A method according to claim 35, wherein said irradiation is emitted by an array of LEDs.

38. A method according to claim 26, wherein the initial temperature of said whole blood sample is between about 18° C. and 34° C.

39. A method according to claim 26, wherein the initial temperature of said whole blood sample is between about 18° C. and 25° C.

40. A method according to claim 26, wherein the initial temperature of said whole blood sample is between about 21° C. and 25° C.

41. A method according to claim 26, wherein said temperature of said whole blood is raised to about 37° C. within 180 seconds.

42. A method according to claim 26, wherein said temperature of said whole blood is raised to about 37° C. within 120 seconds.

43. A method according to claim 26, wherein said temperature of said whole blood is raised to about 37° C. within 90 seconds.

44. A method according to claim 26, wherein said temperature of said whole blood is raised to about 37° C. within 60 seconds.

45. A method according to claim 26, wherein said temperature of said whole blood is raised to about 37° C. within 30 seconds.

46. A method according to claim 26, wherein said method further comprises determining the initial temperature of said whole blood sample.

47. A method according to claim 26, wherein said is configured to operate at an optical power output level of about 1-3 watts.

48. A method according to claim 26, wherein said target temperature of said whole blood sample is determined using an optical sensor.

49. A method according to claim 26, wherein wherein the thickness of the whole blood sample is between about 2 mm and 4 mm.

50. A method for heating a whole blood sample according to claim 49, wherein the thickness of the whole blood sample is about 3.6 mm.

51. An apparatus for heating a whole blood sample contained in a cartridge, comprising:
a housing assembly disposed to receive the cartridge;
one or more visible light optical emitters disposed in the housing apparatus and configured to selectively illuminate a staging area of the cartridge;
a temperature sensor disposed in the housing apparatus and configured to sense a temperature of the cartridge; and
a control circuit coupled to the temperature sensor and the array of visible light optical emitters so as to control a heating profile of the whole blood sample.

52. The apparatus of claim 51 wherein the visible light optical emitters are disposed in an array element.

53. The apparatus of claim 52 wherein the array element comprises a first sub-array element and a second array sub-element and wherein the first sub-array element is positioned so as to be in proximity with a first surface of the cartridge and the second array sub-element is positioned to be in proximity to a second surface of the cartridge.

54. The apparatus of claim 53 wherein the first surface and second surface are on opposite sides of the cartridge.

55. The apparatus of claim 54 wherein the first array sub-element is positioned in proximity to an area of the first surface adjacent to the staging area and the second array sub-element is positioned in proximity to an area of the second surface adjacent to the staging area.

56. The apparatus of claim 55 wherein the first surface and the second surface are on opposite sides of the cartridge.

57. The apparatus of claim 55 wherein the cartridge further comprises a mixing well, and the housing assembly includes a heating element positioned so as to be adjacent to the mixing well when the cartridge is received in the housing assembly.

58. The apparatus of claim 54 wherein the first sub-array element and the second sub-array element are positioned so at to provide substantially equal illumination to the first surface and the second surface.

59. The apparatus of claim 51 wherein the array of optical emitters comprise a plurality of light emitting diode (LED) emitters.

60. The apparatus of claim 59 wherein the plurality of LED optical emitters are red LEDs.

61. The apparatus of claim 59 wherein the plurality of LED optical emitters are high output LEDs.

62. The apparatus of claim 59 wherein the red LEDs are disposed to generate optical output in a range of wavelengths between approximately 600 nanometers and 680 nanometers.

63. The apparatus of claim 59 wherein the red LEDs are disposed to generate optical output in a range of wavelengths between approximately 620 nanometers and 645 nanometers.

64. The apparatus of claim 51 wherein the temperature sensor comprises a non-contact sensor.

65. The apparatus of claim 64 wherein the non-contact sensor is a thermopile.

66. The apparatus of claim 65 wherein the thermopile comprises a PbSe detector.

67. The apparatus of claim 66 wherein the PbSe detector is a cooled detector.

68. The apparatus of claim 65 wherein the thermopile is configured to operate at MWIR wavelengths.

69. The apparatus of claim 65 wherein the thermopile is positioned so as to sense a temperature of the cartridge.

70. The apparatus of claim 65 wherein the thermopile is disposed between two or more emitter elements of the optical array.

71. The apparatus of claim 65 wherein the emitter array comprises a first sub-array and a second sub-array, and the thermopile is positioned adjacent to the first sub-array.

72. The apparatus of claim 71 wherein the first sub-array is positioned adjacent to a first side of the cartridge and the second sub-array is positioned adjacent to a second side of the cartridge, and wherein the first side of the cartridge and the second side of the cartridge are on opposite sides.

73. The apparatus of claim 71 wherein the cartridge comprises a plastic material, and wherein one or more of the optical emitters are selected such that plastic material appears substantially transparent within a wavelength range of the optical emitter output.

74. The apparatus of claim 73 wherein the temperature sensor is a thermopile and the thermopile is selected such that the plastic material is substantially absorptive at the operating wavelength of the thermopile.

75. The apparatus of claim 51 wherein the heating profile comprises heating the whole blood sample from an initial temperature of between approximately 18 and 34 degrees Celsius to a target temperature of approximately 37 degrees Celsius within 120 seconds.

76. The apparatus of claim 51 wherein the control circuit is configured to heat the whole blood sample in accordance with the heating profile.

77. The apparatus of claim 51 wherein the control circuit is configured to perform the steps of:
a. determining an initial temperature of the whole blood sample;
b. selectively adjusting, based at least in part on the initial temperature, the optical output of the array of optical emitters if the initial temperature is below a first predefined threshold;
c. sensing the temperature of the whole blood sample responsive to said selectively pulsing the optical output; and
d. repeating said selectively adjusting and said sensing until the temperature of the whole blood sample reaches a second predefined threshold.

78. The apparatus of claim 77 wherein the first and second predefined thresholds are approximately 35 degrees Celsius.

79. The apparatus of claim 77 wherein the level of optical output of said selectively enabling is set to achieve said second predefined threshold within a predefined time interval.

80. The apparatus of claim 79 wherein said predefined time interval is 120 seconds.

81. The apparatus of claim 51 wherein the control circuit comprises a pulse-width modulation controller.

82. The apparatus of claim 51 wherein the control circuit comprises a bang-bang (on-off) controller.

83. A processor readable medium comprising instructions for execution on a processor to:
determine an initial temperature of a whole blood sample;
optically heat, responsive to said determining, the whole blood sample;
determine an additional temperature of the whole blood sample;
compare said additional temperature to a target temperature; and
repeat said heating, determining and comparing until said additional temperature reaches the target temperature.

84. The medium of claim 83 further comprising instructions to transfer, responsive to said additional temperature reaching said target temperature, said whole blood sample to a mixing well.

85. A processor readable medium comprising instructions for execution on a processor to control the irradiation of a whole blood sample with monochromatic visible light of between 380 nanometers and 740 nanometers until the temperature of the whole blood sample is between approximately 35 degrees Celsius and 40 degrees Celsius.

86. A processor readable medium comprising instructions for execution on a processor to determine a platelet binding function in a whole blood sample comprising controlling the irradiating of said whole blood sample with monochromatic visible light of between 380 nm and 740 nm to raise the temperature of said whole blood sample to a target temperature between about 35° C. and 40° C.; controlling the combining of said whole blood sample with an agglutinating system to form an assay mixture; controlling the irradiating of said assay mixture with infrared light, determining the level of transmission of said infrared light through said assay mixture; wherein said level of transmission or a change in said level of transmission over time is an indication of binding function of said platelets in said whole blood sample.

87. A system for testing whole blood, comprising:
a cartridge disposed to store the whole blood sample in a staging area; and
a testing apparatus comprising:
a housing assembly disposed to receive the cartridge;
one or more visible light optical emitters disposed in the housing apparatus and configured to selectively illuminate the staging area so as to heat the whole blood sample;
a temperature sensor disposed in the housing apparatus and configured to sense a temperature of the cartridge; and
a control circuit coupled to the temperature sensor and the array of visible light optical emitters so as to control said heating of the whole blood sample in order to attain a target temperature.

88. The system of claim 87 wherein the visible light optical emitters are disposed in an array element.

89. The system of claim 88 wherein the array element comprises a first sub-array element and a second array sub-element and wherein the first sub-array element is positioned so as to be in proximity to a first surface of the cartridge and the second array sub-element is positioned to be in proximity to a second surface of the cartridge.

90. The system of claim 89 wherein the first surface and second surface are on opposite sides of the cartridge.

91. The system of claim 89 wherein the first array sub-element is positioned in proximity to an area of the first surface adjacent to the staging area and the second array sub-element is positioned in proximity to an area of the second surface adjacent to the staging area.

92. The system of claim 91 wherein the first surface and the second surface are on opposite sides of the cartridge.

93. The system of claim 91 wherein the cartridge further comprises a mixing well, and the housing assembly includes a heating element positioned so as to be adjacent to the mixing well when the cartridge is received in the housing assembly.

94. The system of claim 90 wherein the first sub-array element and the second sub-array element are positioned so at to provide substantially equal illumination to the first surface and the second surface.

95. The system of claim 87 wherein the array of optical emitters comprise a plurality of light emitting diode (LED) emitters.

96. The system of claim 95 wherein the plurality of LED optical emitters are red LEDs.

97. The system of claim 96 wherein the plurality of LED optical emitters are high output LEDs.

98. The system of claim 96 wherein the red LEDs are disposed to generate optical output in a range of wavelengths between approximately 600 nanometers and 680 nanometers.

99. The system of claim 96 wherein the red LEDs are disposed to generate optical output in a range of wavelengths between approximately 620 nanometers and 645 nanometers.

100. The system of claim 87 wherein the temperature sensor comprises a non-contact sensor.

101. The system of claim 100 wherein the non-contact sensor is a thermopile.

102. The system of claim 101 wherein the thermopile comprises a PbSe detector.

103. The system of claim 102 wherein the PbSe detector is a cooled detector.

104. The system of claim 101 wherein the thermopile is configured to operate at wavelengths that include approximately 9 to 10 micrometers (MWIR wavelengths).

105. The system of claim 101 wherein the thermopile is positioned so as to sense a temperature of the cartridge.

106. The system of claim 101 wherein the thermopile is disposed between two or more visible light emitter elements of the optical array.

107. The system of claim 101 wherein the emitter array comprises a first sub-array and a second sub-array, and the thermopile is positioned adjacent to the first sub-array.

108. The system of claim 107 wherein the first sub-array is positioned adjacent to a first side of the cartridge and the second sub-array is positioned adjacent to a second side of the cartridge, and wherein the first side of the cartridge and the second side of the cartridge are on opposite sides.

109. The system of claim 87 wherein the cartridge comprises a plastic material, and wherein one or more of the optical emitters are selected such that plastic material appears substantially transparent within a wavelength range of the optical emitter output.

110. The system of claim 109 wherein the temperature sensor is a thermopile and the thermopile is selected such that the plastic material is substantially absorptive at the operating wavelength of the thermopile.

111. The system of claim 87 wherein the heating profile comprises heating the whole blood sample from an initial temperature of between approximately 18 and 34 degrees Celsius to a target temperature of approximately 37 degrees Celsius within 120 seconds.

112. The system of claim 87 wherein the control circuit is configured to heat the whole blood sample in accordance with the heating profile.

113. The system of claim 87 wherein the control circuit is configured to perform the steps of:
a. determining an initial temperature of the whole blood sample;
b. selectively adjusting, based at least in part on the initial temperature, the optical output of the array of optical emitters if the initial temperature is below a first predefined threshold;
c. sensing the temperature of the whole blood sample responsive to said selectively pulsing the optical output; and
d. repeating said selectively adjusting and said sensing until the temperature of the whole blood sample reaches a second predefined threshold.

114. The system of claim 113 wherein the first and second predefined thresholds are approximately 35 degrees Celsius.

115. The system of claim 113 wherein the level of optical output of said selectively enabling is set to achieve said second predefined threshold within a predefined time interval.

116. The system of claim 115 wherein said predefined time interval is 120 seconds.

117. The system of claim 87 wherein the control circuit comprises a pulse-width modulation controller.

118. The system of claim 87 wherein the control circuit comprises a bang-bang (on-off) controller.

119. The apparatus of claim 51 wherein the visible light optical emitters are driven by the control circuit to selectively illuminate the staging area in accordance with the heating profile using pulse-width modulation.

120. The apparatus of claim 51 wherein the visible light optical emitters are driven by the control circuit to selectively illuminate the staging area in accordance with the heating profile using amplitude level control of the visible light optical emitters.

121. The system of claim 87 wherein the visible light optical emitters are driven by the control circuit to selectively illuminate the staging area using pulse-width modulation.

122. The system of claim 87 wherein the visible light optical emitters are driven by the control circuit to selectively illuminate the staging area using amplitude level control of the visible light optical emitters.
